(19) United States
(12) Patent Application Publication
(10) Pub. No.: US 2025/0372244 A1
(43) Pub. Date: Dec. 4, 2025

(54) DATA TRANSFER MECHANISM BETWEEN MEDICAL DEVICES FOR MEDICAL DATA GOVERNANCE

(71) Applicant: OneSource Solutions International, Inc., Sudbury, MA (US)

(72) Inventors: Vedran Jukic, 34123 Trieste (TS) (IT); Harold Arkoff, Sudbury, MA (US)

(73) Assignee: OneSource Solutions International, Inc., Sudbury, MA (US)

(21) Appl. No.: 19/011,080

(22) Filed: Jan. 6, 2025

Related U.S. Application Data
(63) Continuation-in-part of application No. 18/677,709, filed on May 29, 2024, now abandoned.

(51) Int. Cl. G16H 40/60; G16H 10/60; G16H 40/20

(52) U.S. Cl. CPC G16H 40/60; G16H 10/60; G16H 40/20

(57) ABSTRACT

A system for data transfer mechanism between medical devices for medical data governance is disclosed. The system receives, from one or more data sources, first medical data associated with a first patient. The first medical data is received using a first proprietary data transmission protocol. The system parses the first proprietary data transmission protocol based on the received first medical data and converts the first proprietary data transmission protocol to a standard data transmission protocol. The system applies one or more pre-processing operations on the first medical data and generates second medical data. The system transmits the second medical data to a medical data governance (MDG) system and receives a first set of commands from the MDG system. The system controls at least one data source to execute the first set of commands.

### FIELD OF TECHNOLOGY

The present disclosure relates generally to data transfer between medical devices, and more specifically to the data transfer mechanism between medical devices for medical data governance.

### BACKGROUND

Modern hospitals are complex, technologically sophisticated organizations having sometimes thousands of employees, doctors, nurses, medical technicians, and administrators, with critical life or death decisions being made regularly and sometimes having to be made abruptly and quickly. Up-to-date, perspicuous, and complete data about the patient is desirable. Even when critical decisions are not at stake, the increase in the cost of health care has made it imperative to use patient data, facility personnel, and resources as efficiently as possible.

Hospitals and other healthcare facilities providing surgical services must coordinate a variety of resources, medical personnel, and hospital staff to provide optimum and efficient care to their patients. Patient data collected during operations, other medical procedures, and patient recovery is updated continually and often needs to be displayed immediately and in an efficient and speedily apprehended manner to attending medical personnel. The patient data is permanently retained in a standard format useful for facility management and medical researchers among others who may be in remote locations and/or need to compare data from different healthcare facilities.

In typical hospital settings such as an operating room, ICU, recovery room, etc., there are multiple medical devices surrounding a patient. In some scenarios, medical data received from one device has to be shared with other devices so that an output can be generated. However, such sharing of medical data between medical devices is complicated and cumbersome because of the different data communication protocols used by the medical devices.

### BRIEF SUMMARY OF THE DISCLOSURE

Systems and/or methods are provided for data transfer mechanisms between medical devices for medical data governance, substantially as shown in and/or described in connection with at least one of the figures, as set forth more completely in the claims.

In accordance with an embodiment, a system for data transfer mechanism between medical devices for medical data governance is provided. The system may receive, from one or more data sources, first medical data associated with a first patient. The first medical data may be received using a first proprietary data transmission protocol. The system may parse the first proprietary data transmission protocol based on the received first medical data. The system may further convert the first proprietary data transmission protocol to a standard data transmission protocol based on the parsing of the first proprietary data transmission protocol. The system may further apply one or more pre-processing operations on the first medical data based on the conversion. The application of the one or more pre-processing operations on the first medical data comprises appending a timestamp to the first medical data. The system may further generate second medical data based on the application of one or more pre-processing operations on the first medical data. The system may further transmit the second medical data, using the standard data transmission protocol, to a medical data governance (MDG) system. The system may further receive a first set of commands from the MDG system based on the transmitted second medical data. The system may further control at least one data source to execute the first set of commands.

In accordance with an embodiment, the one or more data sources comprise at least one of: a set of medical devices, or a set of scanning devices, and wherein the set of medical devices comprises a set of diagnostic medical devices and a set of monitoring devices.

In accordance with an embodiment, the first medical data correspond to at least one of: a medical professional's prescription note, a pathology report, an X-radiation (X-RAY) report, a computed tomography (CT) report, a magnetic resonance imaging (MRI) report, an ultrasound report, a cardiac catheter report, or a cardiac stress report associated with the first patient.

In accordance with an embodiment, the MDG system comprises a set of medical record databases, and wherein a first medical record database of the set of medical record databases is associated with at least one of: the first patient, a first medical condition associated with the first patient, or a first medical facility associated with the first patient.

In accordance with an embodiment, the MDG system comprises one or more artificial intelligence (AI) models. The system may further control the MDG system to apply the one or more AI models on the second medical data. The system may further control the MDG system to generate the first set of commands based on the application of the one or more AI models on the second medical data. The system may further receive the first set of commands from the MDG system.

In accordance with an embodiment, the system may control the MDG system to determine an upcoming medical condition of the first patient based on the application of the one or more AI models on the second medical data. The system may receive the determined upcoming medical condition from the MDG system. The system may render the upcoming medical condition.

In accordance with an embodiment, the system may receive, from the one or more data sources, third medical data associated with the first patient. The second medical data is received after the execution of the first set of commands. The system may transmit the third medical data, using the standard data transmission protocol, to the MDG system. The system may further control the MDG system to apply the one or more AI models on the second medical data, the third medical data, and the first set of commands. The system may further control the MDG system to generate a second set of commands based on the application of the one or more AI models on the second medical data, the third medical data, and the first set of commands. The system may further receive the generated second set of commands based on the application of the one or more AI models on the first medical data, the one or more commands, and the second medical data. The system may further control at least one data source to execute at least one command of the second set of commands.

In accordance with an embodiment, the system may compare the third medical data with pre-defined medical data and transmit an alert to a set of user devices based on the comparison.

In accordance with an embodiment, the system may generate medical metadata associated with the first patient based on the application of one or more pre-processing operations on the first medical data and render the generated medical metadata.

In accordance with an embodiment, the system may generate audit trails based on the first medical data, the second medical data, and the first set of commands and store the generated audit trails.

In accordance with an embodiment, the system may validate the received first set of commands based on one or more criteria and control at least one data source to execute the first set of commands based on the validation.

In accordance with an embodiment, a method for data transfer mechanism between medical devices for medical data governance is provided. The method includes receiving, from one or more data sources, first medical data associated with a first patient. The first medical data may be received using a first proprietary data transmission protocol. The method includes parsing the first proprietary data transmission protocol based on the received first medical data. The method includes converting the first proprietary data transmission protocol to a standard data transmission protocol based on the parsing of the first proprietary data transmission protocol. The method includes applying one or more pre-processing operations on the first medical data based on the conversion. The application of the one or more pre-processing operations on the first medical data comprises appending a timestamp to the first medical data. The method includes generating second medical data based on the application of one or more pre-processing operations on the first medical data. The method includes transmitting the second medical data, using the standard data transmission protocol, to a medical data governance (MDG) system. The method includes receiving a first set of commands from the MDG system based on the transmitted second medical data. The method includes controlling at least one data source to execute the first set of commands.

In accordance with an embodiment, the one or more data sources comprise at least one of: a set of medical devices, or a set of scanning devices, and wherein the set of medical devices comprises a set of diagnostic medical devices and a set of monitoring devices.

In accordance with an embodiment, the first medical data correspond to at least one of: a medical professional's prescription note, a pathology report, an X-radiation (X-RAY) report, a computed tomography (CT) report, a magnetic resonance imaging (MRI) report, an ultrasound report, a cardiac catheter report, or a cardiac stress report associated with the first patient.

In accordance with an embodiment, the MDG system comprises a set of medical record databases, and wherein a first medical record database of the set of medical record databases is associated with at least one of: the first patient, a first medical condition associated with the first patient, or a first medical facility associated with the first patient.

In accordance with an embodiment, the method includes receiving, from the one or more data sources, second medical data associated with the first patient, wherein the second medical data is received after the execution of the first set of commands. The method further includes controlling the MDG system to apply one or more artificial intelligence (AI) models on the second medical data. The method further includes controlling the MDG system to generate the first set of commands based on the application of the one or more AI models on the second medical data, and receiving the first set of commands from the MDG system.

In accordance with an embodiment, the method includes controlling the MDG system to determine an upcoming medical condition of the first patient based on the application of the one or more AI models on the second medical data. The method further includes receiving the determined upcoming medical condition from the MDG system. The method further includes rendering the upcoming medical condition.

In accordance with an embodiment, the method includes receiving, from the one or more data sources, third medical data associated with the first patient. The second medical data is received after the execution of the first set of commands. The method further includes transmitting the third medical data, using the standard data transmission protocol, to the MDG system. The method further includes controlling the MDG system to apply the one or more AI models on the second medical data, the third medical data, and the first set of commands. The method further includes controlling the MDG system to generate a second set of commands based on the application of the one or more AI models on the second medical data, the third medical data, and the first set of commands The method further includes receiving the generated second set of commands based on the application of the one or more AI models on the first medical data, the one or more commands, and the second medical data. The method further includes controlling at least one data source to execute at least one command of the second set of commands.

In accordance with an embodiment, the method includes generating audit trails based on the first medical data, the second medical data, and the first set of commands. The method further includes storing the generated audit trails.

In accordance with an embodiment, a non-transitory computer-readable medium for data transfer mechanism between medical devices for medical data governance is provided. The non-transitory computer-readable medium includes computer program instructions, which when executed by a system, cause the system to perform one or more operations comprising. The operations include receiving, from one or more data sources, first medical data associated with a first patient. The first medical data may be received using a first proprietary data transmission protocol. The operations include parsing the first proprietary data transmission protocol based on the received first medical data. The operations include converting the first proprietary data transmission protocol to a standard data transmission protocol based on the parsing of the first proprietary data transmission protocol. The operations include applying one or more pre-processing operations on the first medical data based on the conversion. The application of the one or more pre-processing operations on the first medical data comprises appending a timestamp to the first medical data. The operations include generating second medical data based on the application of one or more pre-processing operations on the first medical data. The operations include transmitting the second medical data, using the standard data transmission protocol, to a medical data governance (MDG) system. The operations include receiving a first set of commands from the MDG system based on the transmitted second medical data. The operations include controlling at least one data source to execute the first set of commands.

### BRIEF DESCRIPTION OF SEVERAL VIEWS OF THE DRAWINGS

FIG. 1 is a block diagram that illustrates an exemplary environment for data transfer mechanism between medical devices for medical data governance, in accordance with an exemplary embodiment of the disclosure.

FIG. 2 is a block diagram that illustrates an exemplary system for data transfer mechanism between medical devices for medical data governance, in accordance with an embodiment of the disclosure.

FIG. 3 is a diagram that illustrates exemplary operations for data transfer mechanism between medical devices for medical data governance, in accordance with an embodiment of the disclosure.

FIG. 4 is a diagram that illustrates exemplary operations for querying medical record databases, in accordance with an embodiment of the disclosure.

FIG. 5 is a diagram that illustrates a set of data sources for obtaining medical data to be stored in a set of medical record databases, in accordance with an embodiment of the disclosure.

FIG. 6 is a diagram that illustrates an exemplary scenario for an application for medical data governance, in accordance with an embodiment of the disclosure.

FIG. 7 is a flowchart that illustrates an exemplary method for data transfer mechanism between medical devices for medical data governance, in accordance with an embodiment of the disclosure.

FIG. 8 is a conceptual diagram illustrating an example of a hardware implementation for a system used for data transfer mechanism between medical devices for medical data governance, in accordance with an exemplary embodiment of the disclosure.

FIG. 9 is a diagram that illustrates exemplary operations for execution of a set of commands, in accordance with an embodiment of the disclosure.

FIG. 10 is a flowchart that illustrates a method for data transfer mechanism between medical devices for medical data governance, in accordance with an embodiment of the disclosure.

### DETAILED DESCRIPTION OF THE DISCLOSURE

Various aspects of the disclosure may be found in a system and method for data transfer mechanism between medical devices for medical data governance.

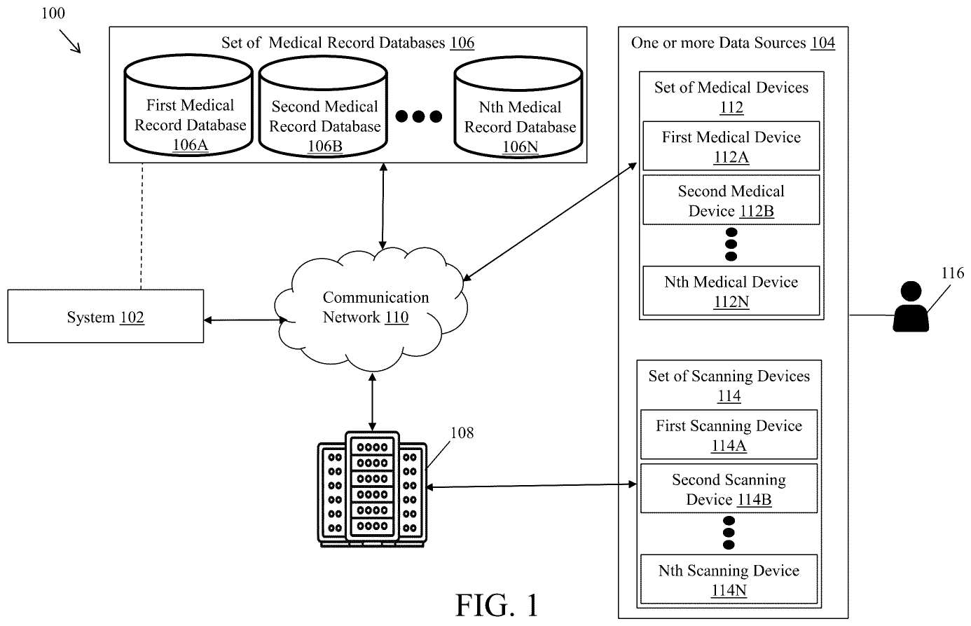

FIG. 1 is a block diagram that illustrates an exemplary environment for data transfer mechanism between medical devices for medical data governance, in accordance with an exemplary embodiment of the disclosure. Referring to FIG. 1, there is shown a network environment 100, which may include a system 102, one or more data sources 104, a set of medical record (MR) databases 106 (or a set of medical data governance (MDG) databases), a server 108, and a communication network 110. The one or more data sources 104 may include a set of medical devices 112, and a set of scanning devices 114. The set of MR databases 106 may include a first MR database 106A, a second MR database 106B, up to an Nth MR database 106N. The set of medical devices 112 may include a first medical device 112A, a second medical device 112B, up to an Nth medical device 112N. Similarly, the set of scanning devices 114 may include a first scanning device 114A, a second scanning device 114B, up to an Nth scanning device 114N. With reference to FIG. 1, there is further shown a first patient 116.

The system 102 may comprise suitable logic, circuitry, interfaces, and/or code that may be configured to receive, from the one or more data sources 104, first medical data associated with the first patient 116. In an embodiment, the first medical data may be received using a first proprietary data transmission protocol. The system 102 may be further configured to convert the first proprietary data transmission protocol to a standard data transmission protocol based on the reception of the first medical data. The system 102 may be further configured to transmit the first medical data using the standard data transmission protocol to generate a first electronic medical record associated with the first patient 116. The first electronic medical record is stored in the first medical record database 106A of a set of medical record databases 106. Examples of the system 102 may include, but are not limited to, a computing device, a mainframe machine, a server, a computer workstation, a smartphone, a cellular phone, a mobile phone, a gaming device, and/or a consumer electronic (CE) device with image processing capabilities.

In an embodiment, the system 102 may enable medical data governance (MDG). The MDG may provide a true source of data that can highlight the schedule of medical treatments and provides tools for rescheduling feedback, contacting and receiving feedback from patients/physicians/healthcare professionals thus introducing general system flexibility through the use of lean process and six sigma methods. MDG leverages modem communication methods (phone apps, emails, web services, etc.) and easily links patient's physicians, or other healthcare professionals to the scheduled use of medical devices. After any unexpected events that may cause a miss in scheduled operations, the MDG may create a backup schedule to pre-emptively fill the gaps and may facilitate healthcare and schedule professionals to optimize machine time usage. This could create a new marketplace for priority services for those patients who opt for it.

Also, MDG may enable patients/users and or institutions to monetize their vital, medically relevant patient data collected during the stay inside the healthcare institution, as well through the extended data collected over some time in multiple stays or spot measurements in healthcare institutions. Patients may be able to establish a relationship with a third party (such as a drug manufacturer, independent drug trial projects, undisclosed trials to the institution) and provide to the third party normalized data collected, organized, and provided by the MDG, and provided to the patient in a different standardized format, even in near real-time. The institution might not be aware of the final user of the patient data. MDG can create additional revenue for the institution by charging such a service per patient and data processed. MDG can track and trace data usage per patient and assets. MDG through the export of all specific, validated clinical data, and medical relevant data, could create a new data-based economy.

Furthermore, MDG manages patient consent and approval, notification for the use of the patient data for second opinions, medical treatments, specific research, validation projects, and educational purposes. Specific patient or user data is screened based on always updated, public, generic, anonymous metadata (for example: sex, age, days in hospital, normalized data content and length: heart rate, respiration rate, drugs, etc.). MDG can handle patient consent using modern communication methods (phone apps, emails, web services, etc.) and provide patient consent for his data to be used in a specific research or validation project, with or without compensation. MDG can provide patient consent and access to the data to specific users, like doctors, physicians, and other specific medical professionals. MDG can provide specific code associated with the data, that, based on necessity, can provide, if granted by the user or proxy consent, protected personal identification, family relations, or other protected personal data

The MDG may allow for third-party statistical analysis (research) on the whole population dataset, without exporting or providing data to the third party, but rather comparing the result to the legally available consent subset group. A statistically relevant result might indicate a minimal group of statistically significant subset of data to search consent and optimize the time for valid and repeatable datasets.

Each of the one or more data sources 104 may correspond to an originator of medical data (such as the first medical data) that may be associated with the first patient 116. Each of the one or more data sources 104 may be configured to capture the first medical data that may be associated with the first patient 116 and further transmit the captured first medical data to the system 102. In an embodiment, the one or more data sources 104 may include the set of medical devices 112, and the set of scanning devices 114. In an alternate embodiment, the one or more data sources 104 may correspond to databases associated with the set of medical devices 112, and the set of scanning devices 114.

Each of the set of medical record databases 106 may correspond to a structured collection of organized information stored electronically in a way that enables easy access, retrieval, and manipulation of medical data. The set of medical record databases 106 may serve as a centralized database where the medical data may be systematically arranged into tables, records, and fields, following a predefined data model. The set of medical record databases 106 may be designed to efficiently manage vast amounts of information, allowing users to perform queries, insert new data, update existing records, and delete information based on specific requirements. In an embodiment, the set of medical record databases 106 may correspond to a storage system associated with the MDG. Examples of different types of the set of medical record databases 106 may include, but are not limited to, a relational database, a non-relational database, a document database, and a graph database.

The server 108 may include suitable logic, circuitry, and interfaces, and/or code that may be configured to store the first medical data. The server 108 may be further configured to store the set of medical record databases 106. The server 108 may be implemented as a cloud server and may execute operations through web applications, cloud applications, HTTP requests, database operations, file transfer, and the like. Other example implementations of the server 108 may include, but are not limited to, a database server, a file server, a web server, a media server, an application server, a mainframe server, or a cloud computing server.

In at least one embodiment, the server 108 may be implemented as a plurality of distributed cloud-based resources by use of several technologies that are well known to those ordinarily skilled in the art. A person with ordinary skill in the art will understand that the scope of the disclosure may not be limited to the implementation of the server 108 and the system 102 as two separate entities. In certain embodiments, the functionalities of the server 108 can be incorporated in its entirety or at least partially in the system 102, without a departure from the scope of the disclosure.

The communication network 110 may include a communication medium through which the system 102, the one or more data sources 104, the set of medical record databases 106, and the server 108 may communicate with each other. The communication network 110 may be one of a wired connection or a wireless connection. Examples of the communication network 110 may include, but are not limited to, the Internet, a cloud network, a Wireless Fidelity (Wi-Fi) network, a Personal Area Network (PAN), a Local Area Network (LAN), or a Metropolitan Area Network (MAN). Various devices in the network environment 100 may be configured to connect to the communication network 110 in accordance with various wired and wireless communication protocols. Examples of such wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity (Li-Fi), 802.16, IEEE 802.11s, IEEE 802.11g, multi-hop communication, wireless access point (AP), device to device communication, cellular communication protocols, and Bluetooth (BT) communication protocols.

Each of the set of medical devices 112 include suitable logic, circuitry, and interfaces, and/or code that may be configured to capture the first medical data that may be associated with the first patient 116. Each of the set of medical devices 112 may correspond to specialized instruments, apparatuses, machines, or implants designed for use in the diagnosis, treatment, monitoring, or prevention of medical conditions of the first patient 116. Examples of the set of medical devices may include, but are not limited to, Vital Sign Monitors, ECG Machines, Pacemakers and Implantable Cardioverter Defibrillators (ICDs), Blood Glucose Monitors, Ultrasound Machines, and X-ray machines.

Each of the set of scanning devices 114 include suitable logic, circuitry, and interfaces, and/or code that may be configured to capture the first medical data that may be associated with the first patient 116. Specifically, each of the set of scanning devices 114 may correspond to specialized instruments used for imaging and diagnosis and may be configured to capture one or more images of the first patient 116. Such one or more images may correspond to the first medical data associated with the first patient 116. The set of scanning devices 114 may employ various technologies such as electromagnetic waves or sound waves to capture the first medical data. Examples of the set of scanning devices 114 may include, but are not limited to, MRI (Magnetic Resonance Imaging) Scanners, CT (Computed Tomography) Scanners, PET (Positron Emission Tomography) Scanners, and Ultrasound Scanners.

In operation, the system 102 may be configured to receive the first medical data associated with the first patient 116. In an embodiment, the first medical data may be received from the one or more data sources 104. As an example, the first medical data may be received from the first medical device 112A of the set of medical devices 112. As discussed above, the set of medical devices 112 may be included in the one or more data sources 104. In an embodiment, the system 102 may be configured to receive the first medical data from the set of medical devices 112 and the set of scanning devices 114 wirelessly. In such implementation, each of the set of medical devices 112 and the set of scanning devices 114 may be configured to wirelessly transmit the captured first medical data to the system 102.

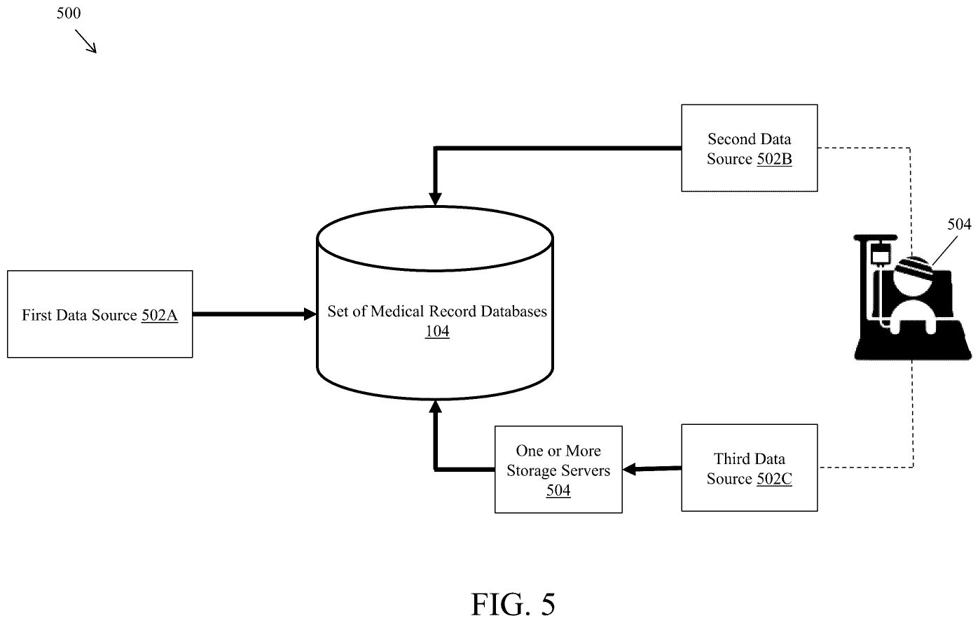

In an alternate implementation, each of the set of scanning devices 114 may be configured to transmit the captured first medical data to the server 108. In such an implementation, the server 108 may correspond to a storage server (for e.g., a Picture Archiving and Communication System (PACS) server). The server 108 may be configured to receive the first medical data from the set of scanning devices 114 and further transmit the received first medical data to the set of the MR databases 106. In the set of MR databases 106, the first medical data may be stored as an electronic medical record (EMR). In the set of MR databases 106, metadata may be associated with the EMR. Further, the metadata along with the EMR may be transmitted to the system 102 via the communication network 108 for further processing as discussed below. Details about the metadata are provided, for example, in FIG. 5.

Based on the reception of the first medical data, the system 102 may be further configured to convert the first proprietary data transmission protocol to a standard data transmission protocol. In an embodiment, a data transmission protocol (such as the first proprietary data transmission protocol, or the standard data transmission protocol) may correspond to a set of rules, procedures, and conventions that govern the exchange of data between the system 102 and the one or more data sources 104 in the network environment 100. The data transmission protocol may define how data is formatted, transmitted, received, and interpreted, ensuring reliable and efficient communication. The data transmission protocol may specify parameters such as data encoding, error detection and correction mechanism, flow control, synchronization, addressing, and routing. They enable devices to establish connections, exchange information, and synchronize their operations, regardless of differences in hardware, software, or network configurations.

The system 102 may be further configured to transmit the first medical data using the standard data transmission protocol to generate the first electronic medical record associated with the first patient 116. The first electronic medical record may be stored in the first medical record database 106A of the set of medical record databases 106.

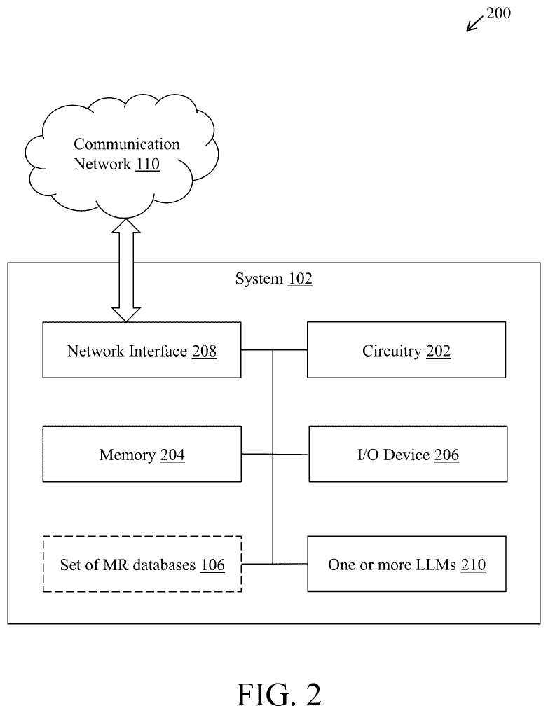

FIG. 2 is a block diagram that illustrates an exemplary system for data transfer mechanism between medical devices for medical data governance, in accordance with an embodiment of the disclosure. FIG. 2 is explained in conjunction with elements from FIG. 1. With reference to FIG. 2, there is shown a block diagram 200 of the system 102. The system 102 may include a circuitry 202, a memory 204, an input/output (I/O) device 206, a network interface 208, one or more LLMs 210, and the set of medical record databases 106. The circuitry 202 may be communicatively coupled to the memory 204, the I/O device 206, the network interface 208, the one or more LLMs 210, and the set of medical record databases 106.

The circuitry 202 may include suitable logic, circuitry, and interfaces that may be configured to execute program instructions associated with different operations to be executed by the system 102. For example, some of the operations may include, but are not limited to, receiving the first medical data, converting the first proprietary data transmission protocol to the standard data transmission protocol, and transmitting the first medical data. The circuitry 202 may include one or more specialized processing units, which may be implemented as an integrated processor or a cluster of processors that perform the functions of the one or more specialized processing units, collectively. The circuitry 202 may be implemented based on a number of processor technologies known in the art. Examples of implementations of the circuitry 202 may be an x86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other computing circuits.

The memory 204 may include suitable logic, circuitry, interfaces, and/or code that may be configured to store the program instructions to be executed by the circuitry 202. In at least one embodiment, the memory 204 may store the first medical data. In an embodiment, the memory 204 may be further configured to store second medical data, the first electronic medical record, and second electronic medical record. Examples of implementation of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 206 may include suitable logic, circuitry, and interfaces that may be configured to receive one or more user inputs and provide an output. For example, the system 102 may receive the user input via the I/O device 206. The I/O device 206 may further display the first electronic medical record and/or the second medical record. The I/O device 206 which includes various input and output devices, may be configured to communicate with the circuitry 202. Examples of the I/O device 206 may include, but are not limited to, a touch screen, a keyboard, a mouse, a joystick, a microphone, a display device, and a speaker.

The network interface 208 may include suitable logic, circuitry, and interfaces that may be configured to facilitate a communication between the circuitry 202, the one or more data sources 104, the set of MR databases 106, and the server 108, via the communication network 110. The network interface 208 may be implemented by use of various known technologies to support wired or wireless communication of the system 102 with the communication network 110. The network interface 208 may include, for example, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 208 may be configured to communicate via wireless communication with networks, such as the Internet, an Intranet, or a wireless network, such as a cellular telephone network, a public switched telephonic network (PSTN), a radio access network (RAN), a wireless local area network (LAN), and a metropolitan area network (MAN). The wireless communication may use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VoIP), light fidelity (Li-Fi), Worldwide Interoperability for Microwave Access (Wi-MAX), a protocol for email, instant messaging, and a Short Message Service (SMS).

Each of the one or more LLMs 210 may correspond to a sophisticated artificial intelligence (AI) system trained on vast amounts of text data, capable of understanding, generating, and processing human-like language at an extensive scale. Each of the one or more LLMs 210 models utilizes deep learning techniques, particularly transformer architectures, enabling them to grasp context, syntax, semantics, and even nuances in language usage. The primary function of the one or more LLMs 210 may involve, but is not limited to, natural language processing tasks like text generation, translation, summarization, and sentiment analysis. Each of the one or more LLMs 210 may learn to predict and generate text by analysing patterns and relationships within the massive corpus of text they've been trained on. Examples of different types of the one or more LLMs 210 may include, but are not limited to, a Transformer-Based Model, a Bidirectional Encoder Representations from Transformers (BERT) model, a Generative Pre-trained Transformer (GPT) model, a Unified Language Model, and a Text-to-Text Transfer Transformer (T5) model.

In an embodiment, each of the one or more LLMs 210 may include a neural network that may be a computational network or a system of artificial neurons, arranged in a plurality of layers, as nodes. The plurality of layers of each of the neural network may include an input layer, one or more hidden layers, and an output layer. Each layer of the plurality of layers may include one or more nodes (or artificial neurons). Outputs of all nodes in the input layer may be coupled to at least one node of hidden layer(s). Similarly, inputs of each hidden layer may be coupled to outputs of at least one node in other layers of the corresponding neural network. Outputs of each hidden layer may be coupled to inputs of at least one node in other layers of the corresponding neural network. Node(s) in the final layer may receive inputs from at least one hidden layer to output a result. The number of layers and the number of nodes in each layer may be determined from hyper-parameters of the corresponding neural network model. Such hyper-parameters may be set before or while training the corresponding neural network model on a training dataset.

The neural network may correspond to a mathematical function (for example, a sigmoid function or a rectified linear unit) with a set of parameters, tuneable during training of the network. The set of parameters may include, for example, a weight parameter, a regularization parameter, and the like. Each node may use the mathematical function to compute an output based on one or more inputs from nodes in other layer(s) (for example, previous layer(s)) of the corresponding neural network model. All or some of the nodes of the each of the set of neural network models may correspond to the same or different mathematical function.

In training of the neural network, one or more parameters of each node of the corresponding neural network may be updated based on whether an output of the final layer for a given input (from the training dataset) matches a correct result based on a loss function for the corresponding neural network. The above process may be repeated for the same or a different input until a minima of loss function may be achieved, and a training error may be minimized. Several methods for training are known in the art, for example, gradient descent, stochastic gradient descent, batch gradient descent, gradient boost, meta-heuristics, and the like.

Each of the set of neural networks may include electronic data, such as a software program, code of the software program, libraries, applications, scripts, or other logic or instructions for execution by a processing device, such as a hardware processor. Each of the set of neural network models may include code and routines configured to enable a computing device, such as the system 102, to perform one or more operations. Additionally or alternatively, the neural network may be implemented using hardware including a processor, a microprocessor, a field-programmable gate array (FPGA), or an application-specific integrated circuit (ASIC) to perform or control the performance of one or more operations. Alternatively, in some embodiments, each of the neural network models may be implemented using a combination of hardware and software.

Although in FIG. 2, the one or more LLMs 210 are shown as integrated within the system 102, the disclosure is not so limited. Accordingly, in some embodiments, the one or more LLMs 210 may be associated with the system 102, without deviation from the scope of the disclosure. In an embodiment, the one or more LLMs 210 may be stored in the server 108.

In an embodiment, the system 102 may correspond to a plug-and-play dongle (such as a universal serial bus (USB) dongle) that may include the circuitry 202, the memory 204, the input/output (I/O) device 206, and the network interface 208. The plug-and-play dongle may have processing capabilities that may include receiving the first medical data associated with the first patient, converting the first proprietary data transmission protocol to the standard data transmission protocol, and transmitting the first medical data using the standard data transmission protocol to generate the first electronic medical record associated with the first patient. In another embodiment, the plug-and-play dongle may be connected to an external computing apparatus (not shown in Fig.) to enhance its data processing capabilities for executing various functions or operation described herein.

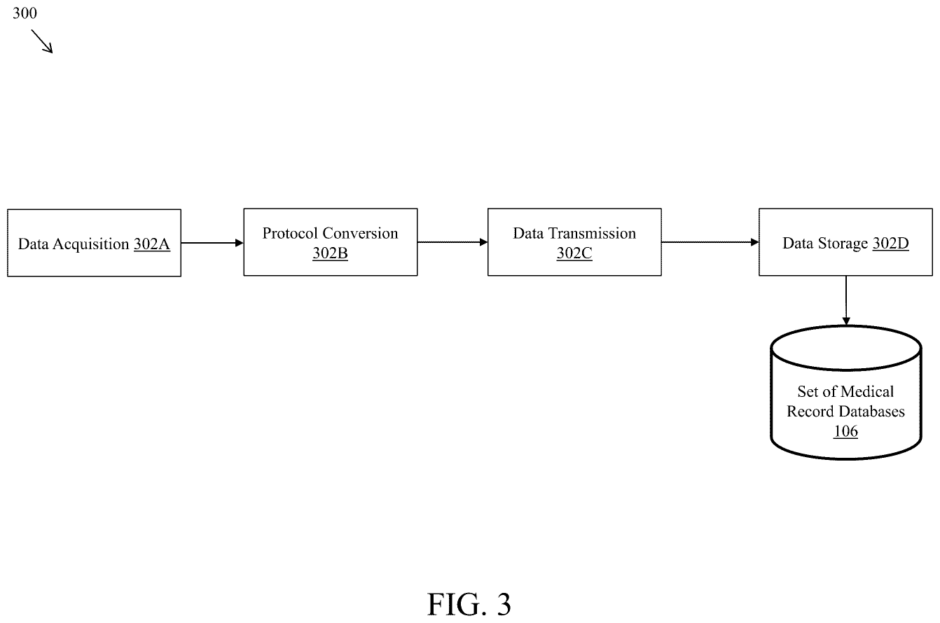

The functions or operations executed by the system 102, as described in FIG. 2, may be performed by the circuitry 202. Various operations executed by the circuitry 202 are described in detail, for example, in FIGS. 3, 4, 5, 6, and 7.

FIG. 3 is a diagram that illustrates exemplary operations for data transfer mechanism between medical devices for medical data governance, in accordance with an embodiment of the disclosure. FIG. 3 is explained in conjunction with elements from FIG. 1 and FIG. 2. With reference to FIG. 3, there is shown a block diagram 300 that illustrates exemplary operations from 302A to 302D, as described herein. The exemplary operations illustrated in the block diagram 300 may start at 302A and may be performed by any computing system, apparatus, or device, such as by the system 102 of FIG. 1 or circuitry 202 of FIG. 2. Although illustrated with discrete blocks, the exemplary operations associated with one or more blocks of the block diagram 300 may be divided into additional blocks, combined into fewer blocks, or eliminated, depending on the particular implementation.

At 302A, a data acquisition operation may be performed. In data acquisition operation, the circuitry 202 may be configured to receive the first medical data. The first medical data may be associated with the first patient 116. In an embodiment, the first patient 116 may be suffering from a medical condition (say a disease) and may be admitted to a hospital. The set of medical devices 112 may be configured to capture the first medical data associated with the first patient 116. In an embodiment, the set of medical devices 112 may further include a set of diagnostic medical devices and a set of monitoring devices.

Each of the set of diagnostic medical devices may correspond to instruments or tools that may be used by healthcare professionals to identify and diagnose the medical conditions in the first patient 116. Each of the set of diagnostic medical devices may often provide quantitative or qualitative data about the health status of the first patient 116. Such data may be useful in determining an appropriate course of treatment for the medical condition. In an embodiment, the set of diagnostic medical devices may include, but are not limited to, a blood glucose monitor, an electrocardiogram (ECG or EKG) machine, a spirometer, and a sphygmomanometer.

Each of the set of monitoring devices may correspond to instruments that may be designed to observe and track various physiological parameters or medical conditions in the first patient 116. Each of the set of monitoring devices may provide real-time data, enabling healthcare professionals to make informed decisions about patient care and treatment adjustments. Examples of such monitoring devices may include, but are not limited to, a pulse oximeter, a glucose monitor, a Holter monitor, a blood pressure monitor, a cardiac monitor, a respiratory rate monitor, a sleep pane monitor, an intracranial pressure monitor, and a wearable fitness tracker.

In an embodiment, the first medical data may be received from the set of scanning devices. Each of the set of scanning devices may correspond to equipment that may be used in healthcare settings to obtain detailed images or scans of internal structures within the human body for diagnostic or monitoring purposes. Examples of the set of scanning devices may include, but are not limited to, an X-ray machine, an MRI (Magnetic Resonance Imaging) scanner, a CT (Computed Tomography) scanner, an ultrasound machine, a Positron Emission Tomography (PET) Scanner, a Single-Photon Emission Computed Tomography (SPECT) Scanner, a Mammography Machine, and an ultrasound machine.

In an embodiment, the system 102 may be configured to receive the first medical data associated with the first patient 116 from the first medical device 112A of the set of medical devices 112 or the first scanning device 114A of the set of scanning devices 114. As discussed above, both the set of medical devices 112 and the set of scanning devices 114 may be included in the one or more data sources 104. In an embodiment, the first medical data may correspond to at least one of a medical professional's prescription note, a pathology report, an X-radiation (X-RAY) report, a computed tomography (CT) report, a magnetic resonance imaging (MRI) report, an ultrasound report, a cardiac catheter report, or a cardiac stress report associated with the first patient 116.

In an embodiment, the first medical device 112A (or the first scanning device 114A) may use the first proprietary data transmission protocol to transmit the data from the first medical device 112A (or the first scanning device 114A) to the system 102. Therefore, the system 102 may receive the first medical data using the first proprietary data transmission protocol.

In an embodiment, the system 102 may be configured to receive the first medical data from one or more storage servers associated with the one or more data sources 104. In such an implementation, the system 102 may be configured to receive the first medical data using one or more application programming interface (API) calls. By way of example and not limitation, each of the set of scanning devices 114 may be configured to transmit the first medical data (that may include images (say X-ray images)) to the one or more storage servers. In an implementation, the one or more storage servers may correspond to a Picture Archiving and Communication System (PACS) server. Further, the system 102 may transmit a Fast Healthcare Interoperability Resources (FHIR) API call to the PACS server. Based on the reception of the FHIR API call, the PACS server may transmit the first medical data to the system 102.

In another embodiment, the first medical data stored in the PACS server may be transmitted to the set of MR databases 106 using the FHIR API call. In the set of MR databases, metadata associated with the first patient may be embedded within the first medical data. Such metadata may correspond to the medical data that may be associated with the first patient. In an embodiment, the medical data may have been captured by the set of medical devices 112 or the set of scanning devices 114. By way of example and not limitation, such metadata may include, but is not limited to, doctor's notes, lab reports, and the like. The system 102 may receive the first medical data along with the metadata from the set of MR databases 106. In an embodiment, the doctor's notes may not be scanned and may be retrieved via the FHIR API call to the set of MR databases 106 where the doctor's note may be stored when dictated by the doctor.

In an alternate embodiment, the system 102 may be configured to receive the first medical data from the set of medical devices 112 and the set of scanning devices 114 wirelessly. In such implementation, each of the set of medical devices 112 and the set of scanning devices 114 may be configured to wirelessly transmit the captured first medical data to the system 102.

At 302B, a protocol conversion operation may be executed. In the protocol conversion operation, the system 102 may be configured to convert the first proprietary data transmission protocol to a standard data transmission protocol based on the reception of the first medical data. In an embodiment, the standard data transmission protocol may be used by the set of medical record databases 106.

In an embodiment, the first medical data may have to be transferred to the other medical devices (say the second medical device 112B) that may use a second proprietary data transmission protocol. In such an embodiment, the system 102 may be further configured to convert the standard data transmission protocol to the second proprietary data transmission protocol before transmission.

At 302C, a data transmission operation may be executed. In the data transmission operation, the system 102 may be configured to transmit the first medical data using the standard data transmission protocol to generate the first electronic medical record. The first electronic medical record may be associated with the first patient 116. In an embodiment, the first electronic medical record may be similar to the first medical data but may be formatted in a different version to be used by the medical device or to be stored in the set of MR databases 106.

In an embodiment, an electronic medical record (EMR) may correspond to digital versions of the paper charts in a healthcare provider's office. Such EMRs may include a patient's medical history, diagnoses, medications, treatment plans, immunization dates, allergies, radiology images, laboratory test results, and the like. In an embodiment, the EMR may include medical history reports, physical examination reports, diagnostic reports, progress notes, consultation reports, operative reports, discharge summaries, medication reports, billing reports, and insurance reports. Examples of the EMRs may correspond to at least one of the doctor consultations notes, doctor progress notes, nurse notes, a prescription history, problem lists, International Classification of Diseases (ICD) codes, laboratory results, pathology reports, X-radiation (X-RAY) reports, computed tomography (CT) reports, magnetic resonance imaging (MRI) reports, ultrasound reports, cardiac catheter reports, or cardiac stress reports associated with the first patient 116.

At 302D, a data storage operation may be executed. In the data storage operation, the system 102 may be configured to store the first electronic medical record in a first medical record database 106A of the set of medical record databases 106. In an embodiment, the first medical record database 106A may be associated with at least one of the first patient 116, a first medical condition associated with the first patient 116, or a first medical facility associated with the first patient 116.

In an embodiment, each MR database of the set of MR databases 106 may include electronic medical records associated with at least one user. In an embodiment, the EMRs may allow for systematic storage, retrieval, and modification of patient data, making it easily accessible to authorized healthcare providers. In an embodiment, the set of MR databases 106 may correspond to a specific storage and retrieval tool that can work very efficiently with the one or more LLMs 210 to create an interface and a communication tool for doctors to synthesize responses based on context and clarity.

In another embodiment, the set of MR databases 106 may further include records associated with multiple patients, medical facilities, and medical conditions (or procedures). The medical facilities encompass diverse settings designed to provide healthcare services and support to individuals. The medical facilities may include, but are not limited to, hospitals, clinics, urgent care centres, rehabilitation centres, and nursing homes. The medical procedures may encompass a vast range of interventions performed by healthcare professionals to diagnose, treat, or prevent various health conditions. Such medical procedures may include diagnostic procedures (such as X-rays, CT scans, MRI scans, ultrasounds, and PET scans), surgical procedures (such as laparoscopy, arthroscopy), therapeutic procedures (such as chemotherapy, and radiation therapy), cardiovascular procedures (such as angioplasty, coronary artery bypass grafting (CABG)), obstetric and gynaecological procedures (such as caesarean section, colposcopy), orthopaedic procedures (such as joint replacement, fracture repair), and dental procedures (such as fillings and root canals, and extractions).

In an embodiment, the system 102 may be configured to receive second medical data associated with the first patient 116. The second medical data associated with the first patient 116 may be received from the one or more data sources 104. In an embodiment, the second medical data may be received using a second proprietary data transmission protocol. The system 102 may be further configured to convert the second proprietary data transmission protocol to the standard data transmission protocol based on the reception of the second medical data. The system 102 may be further configured to transmit the second medical data using the standard data transmission protocol to generate a second electronic medical record associated with the first patient 116. In an embodiment, the first electronic medical record may be stored in the first medical record database 106A of the set of medical record databases 106.

In another embodiment, the system 102 may be configured to transmit the generated first electronic medical record and the second electronic medical record to the Nth medical device 112N of the set of medical devices 112 (or to the Nth scanning device 114N). The Nth medical device 112N may utilize the first electronic medical record and the second electronic medical record to generate Nth medical data associated with the first patient 116. The Nth medical device 112N may be configured to transmit the Nth medical data associated with the first patient 116 to the system 102 using the Nth proprietary data transmission protocol. The system 102 may be further configured to convert the Nth proprietary data transmission protocol to the standard data transmission protocol based on the reception of the Nth medical data. The system 102 may be further configured to transmit the Nth medical data using the standard data transmission protocol to generate an Nth electronic medical record associated with the first patient 116. The Nth electronic medical record is stored in the Nth medical record database 160N of the set of medical record databases 106.

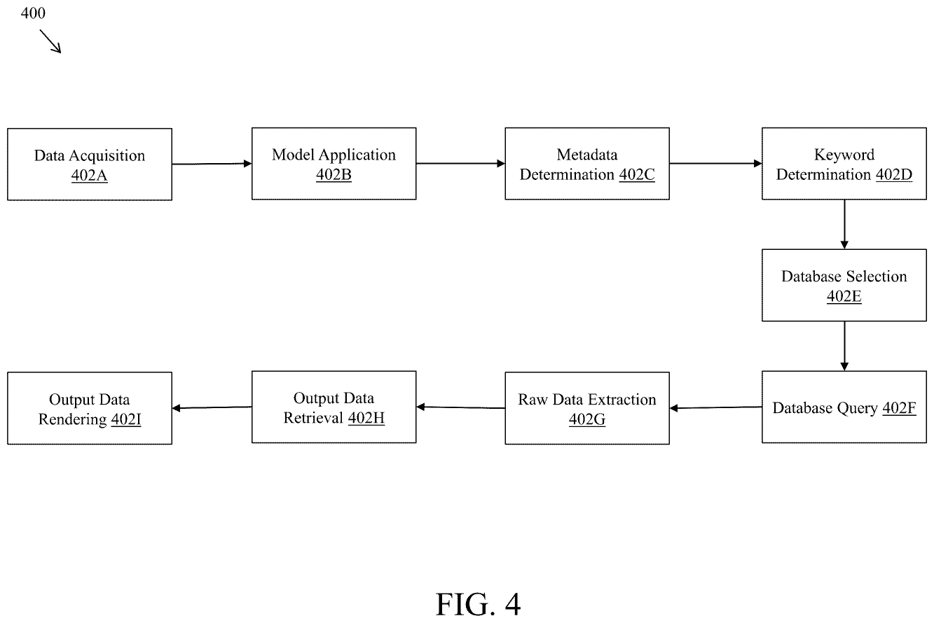

FIG. 4 is a diagram that illustrates exemplary operations for querying medical record databases, in accordance with an embodiment of the disclosure. FIG. 4 is explained in conjunction with elements from FIG. 1, FIG. 2, and FIG. 3. With reference to FIG. 4, there is shown a block diagram 400 that illustrates exemplary operations from 402A to 402I, as described herein. The exemplary operations illustrated in the block diagram 400 may start at 402A and may be performed by any computing system, apparatus, or device, such as by the system 102 of FIG. 1 or circuitry 202 of FIG. 2. Although illustrated with discrete blocks, the exemplary operations associated with one or more blocks of the block diagram 400 may be divided into additional blocks, combined into fewer blocks, or eliminated, depending on the particular implementation.

At 402A, a data acquisition operation may be performed. In data acquisition operation, the circuitry 202 may be configured to receive the user input from a user of the system 102. The user may be, for example, a doctor, a physician, or any medical professional in a medical environment. In an embodiment, the user may be the first patient 116 or any person from the general public. In an embodiment, the user input may be received from a user device associated with the user and via the communication network 110. The user input may include at least one search query that may be written in a first language (say English). As a first example, the at least one search query may be “Does the first patient have the possibility of Sepsis?”

In an embodiment, the at least one search query may be received to retrieve the first electronic medical record from the set of MR databases 106. Each MR database of the set of MR databases 106 may include electronic medical records associated with the first patient 116. The electronic medical records (EMRs) may correspond to digital versions of the paper charts in a healthcare provider's office. Such EMRs may include a patient's medical history, diagnoses, medications, treatment plans, immunization dates, allergies, radiology images, laboratory test results, and the like. In an embodiment, the EMRs may allow for systematic storage, retrieval, and modification of patient data, making it easily accessible to authorized healthcare providers. In an embodiment, the set of MR databases 106 may correspond to a specific storage and retrieval tool that can work very efficiently with the one or more LLMs 210 to create an interface and a communication tool for doctors to synthesize responses based on context and clarity.

In an embodiment, the EMRs may include medical history reports, physical examination reports, diagnostic reports, progress notes, consultation reports, operative reports, discharge summaries, medication reports, billing reports, and insurance reports. Examples of the EMRs may correspond to at least one of the doctor consultations notes, doctor progress notes, nurse notes, a prescription history, problem lists, International Classification of Diseases (ICD) codes, laboratory results, pathology reports, X-radiation (X-RAY) reports, computed tomography (CT) reports, magnetic resonance imaging (MRI) reports, ultrasound reports, cardiac catheter reports, or cardiac stress reports associated with at least one user.

In another embodiment, the set of MR databases 106 may further include records associated with multiple patients, medical facilities, and medical procedures. The medical facilities encompass diverse settings designed to provide healthcare services and support to individuals. The medical facilities may include, but are not limited to, hospitals, clinics, urgent care centres, rehabilitation centres, and nursing homes. The medical procedures may encompass a vast range of interventions performed by healthcare professionals to diagnose, treat, or prevent various health conditions. Such medical procedures may include diagnostic procedures (such as X-rays, CT scans, MRI scans, ultrasounds, and PET scans), surgical procedures (such as laparoscopy, and arthroscopy), therapeutic procedures (such as chemotherapy, and radiation therapy), cardiovascular procedures (such as angioplasty, coronary artery bypass grafting (CABG)), obstetric and gynaecological procedures (such as caesarean section, colposcopy), orthopaedic procedures (such as joint replacement, fracture repair), and dental procedures (such as fillings and root canals, and extractions).

At 402B, a model application operation may be executed. In the model application operation, the circuitry 202 may be configured to apply the one or more LLMs 210 on the received at least one search query. As discussed above, each of the one or more LLMs 210 may be pre-trained models that may be trained to extract metadata based on the received at least one search query. In an embodiment, the one or more LLMs 210 may be been trained on all medical textbooks that may be known in the art. As an example, the one or more LLMs 210 may put together about 65 billion words and may be used to provide all metadata needed for successful research and then provide it back as an interface to humans. Specifically, the one or more LLMs 210 may be used as an interface for the research.

At 402C, a metadata determination operation may be executed. In the metadata determination operation, the circuitry 202 may be configured to determine metadata associated with the at least one search query. In an embodiment, the metadata may be determined based on the application of the one or more LLMs 210 on the received search query. The metadata may correspond to information about the data, such as the type of data, the date it was collected, and the patient it is associated with. By indexing the metadata, users (such as doctors) may be able to search for data across MDG databases without having to access the data itself.

In an embodiment, the metadata may be required for successful research. Specifically, the one or more LLMs 210 may be used to provide all metadata that are needed for successful research and provide it back as an interface to the user. With reference to the first example, the one or more LLMs 210 by itself may find the metadata needed to define sepsis and determine whether the first patient may have sepsis based on the medical data associated with the first patient 116 that may be captured by the set of medical devices 112 and/or the set of scanning devices 114.

At 402D, a keyword determination operation may be executed. In the keyword determination operation, the circuitry 202 may be configured to determine at least one keyword from the determined metadata. In an embodiment, the least one keyword is associated with the at least one search query. In an embodiment, the at least one keyword corresponds to one of a name of the first patient 116, a name of a medical facility in which the first patient 116 is admitted, a name of a disease from which the first patient 116 may be suffering, or a name of a medical procedure to be done on the first patient 116.

In an embodiment, the system 102 may be configured to apply the one or more LLMs 210 on the determined metadata to further determine the at least one keyword. In another embodiment, the system 102 may be configured to apply a natural language processing (NLP) model on the metadata to determine the at least one keyword. With reference to the first example, the at least one keyword may be “Sepsis”.

At 402E, a database selection operation may be executed. In the database selection operation, the circuitry 202 may be configured to select the first MR database 106A of the set of MR databases 106 based on the determined at least one keyword. In an embodiment, each of the set of MR databases 106 may be associated with at least one keyword. For example, the first MR database 106A may include all the information about the patients associated with at least one medical disease (such as sepsis), the second MR database 106B may include medical records associated with the set of patients in a geographic area (such as a city or a town), the Nth MR database 106N may include details about all the patients, medical equipment, facilities available in at least one medical clinic in the geographic area, and so on.

At 402F, a database query operation may be executed. In the database query operation, the circuitry 202 may be configured to query the selected first MR database 106A of the set of MR databases 106. In an embodiment, querying the first MR database 106A may refer to a process of requesting specific information or data from the first MR database 106A. The system 102 may be configured to query at least the first MR database 106A of the set of MR databases 106 to determine at least one upcoming event associated with the first medical condition of the first patient 116 based on querying the at least one medical record database and the received first medical data from the one or more data sources 104. The received first medical data may correspond to real-time medical data associated with the first patient. In an embodiment, the utilization of real-time medical data, the disclosed system 102 may be configured to identify patterns, trends, and anomalies in vital parameters of the first patient, thereby facilitating early detection and intervention of at least one of the upcoming events. By using the real-time data, the disclosed system 102 may further empower personalized medicine by tailoring treatments to the needs of the first patient, thereby optimizing therapeutic efficacy, and minimizing adverse effects.

At 402G, a raw data extraction operation may be executed. In the raw data retrieval operation, the circuitry 202 may be configured to extract, from the first MR database 106A, raw data associated with the determined metadata based on the querying the first MR database 106A. The raw data may include all the data extracted from at least the first MR database 104A that may be associated with the determined at least one keyword. With reference to the first example, the raw data may include a knowledge base associated with the medical disease sepsis, a possibility of the first patient suffering from sepsis, details associated with one or more patients suffering from sepsis in the geographical area, medical facilities offering treatment for sepsis, trends in the people suffering from sepsis, and the like.

At 402H, an output data retrieval operation may be performed. In the output data retrieval operation, the circuitry 202 may be configured to retrieve the output data. In an embodiment, the system 102 may be configured to retrieve the output data from the extracted raw data. To retrieve the output data, the system 102 may be configured to generate one or more constructs associated with the one or more LLMs 210 to be applied on the extracted raw data. The one or more constructs may be associated with the determined metadata. The system 102 may be further configured to retrieve the output data based on the application of the generated one or more constructs on the extracted raw data. The first medical data may correspond to an appropriate answer to the at least one search query and maybe a desired answer to the at least one search query.

With reference to the first example, the output data may be indicative of whether the first patient is suffering from sepsis or has a possibility of developing sepsis in the near future.

At 402I, an output data rendering operation may be performed. In the output data rendering operation, the circuitry 202 may be configured to render the determined at least one upcoming event on the user device associated with the medical professional. In an embodiment, the rendering of the output data may correspond to displaying the output data on the user device. In another embodiment, the rendering of the output data may correspond to storing the output data in the set of MR databases 106.

In an embodiment, the disclosed system may be configured to create thousands of descriptions (combinations and permutations of data elements) results using metadata from an LLM Query, from the first medical data governance database. This structured data, associated with the determined metadata, may be expressed as a configuration to LLM in the form of specific language terms and sentences used to train the LLM (English or German, for example). The LLM query may further create a specific syntax for medical data governance, based on the context of the question and previous queries.

In another embodiment, the system 102 (or the MDG system) may enable federated search of medical data. The federated search may allow users to search for data across multiple distributed systems without having to centralize the data in one location. This may be important for protecting the privacy of patients and for reducing the administrative burden on hospitals.

There may be several different approaches to the federated search of medical data. One important approach of MDG is to generate metadata to index the sensitive data. As discussed above, the metadata is information about the data, such as the type of data, the date it was collected, and the patient it is associated with. By indexing the metadata, the users may be able to search for data across multiple systems without having to access the data itself.

Another advantage of the MDG may be that it acts as a centralized storage system for different healthcare systems or medical facilities (such as hospitals) that may be accessed using a query mediator. The query mediator (or the one or more LLMs) may be a component that may acts as an intermediary between the user and the distributed MDG subsystems. The query mediator component receives the user's search query and forwards it to the relevant MDG subsystems, providing unifying results back to the user.

Some specific examples of how federated search of medical data is used may include a researcher search for data on patients with a particular disease across multiple hospitals. This could help the researcher to identify new risk factors for the disease or to develop new treatments. Another example may be that a clinician may search for data on patients who have had a particular surgery across multiple hospitals. This could help the clinician to learn from the experiences of other surgeons and to improve their surgical outcomes. Another example may be that a patient may use a search for data on their medical history across multiple hospitals. This could help the patient to better understand their health and to make informed decisions about their care.

Therefore, MDG federated search of medical data may be a powerful tool that may be used to improve patient care and advance medical research. MDG may be essential for enabling federated search in a secure and privacy-preserving manner.

In addition to these general benefits, MDG can also provide a number of specific benefits for research, such as facilitating data sharing, supporting the development of new research methods, and promoting the translation of research findings into clinical practice. Specifically, MDG may facilitate data sharing between researchers, which can help to accelerate research and improve the quality and impact of research findings. MDG may also support the development of new research methods, such as the use of machine learning and artificial intelligence to analyse large datasets. Furthermore, MDG may also help to promote the translation of research findings into clinical practice by ensuring that data is collected and stored in a way that is compatible with clinical systems.

The specific value of MDG for research on patient medical data is that it may allow researchers to access and use large datasets of patient data without compromising the privacy of individual patients. This is possible because MDG may ensure that data is de-identified before it is used for research. Such de-identified patient medical data may be used to conduct a wide range of research studies, including clinical trials, Observational studies, and Basic science research.

Therefore, MDG may be essential for ensuring that medical data is used responsibly and ethically for research. This is important for protecting the privacy of patients, ensuring the quality of data, promoting the ethical use of data, and accelerating the development of new medical treatments and cures.

FIG. 5 is a diagram that illustrates a set of data sources for obtaining medical data to be stored in a set of medical record databases, in accordance with an embodiment of the disclosure. FIG. 5 is explained in conjunction with elements from FIGS. 1, 2, 3, and 4. With reference to FIG. 5, there is shown a diagram 500 that includes one or more data sources for obtaining the medical data to be stored in the set of MR databases 106. In an embodiment, the one or more data sources may include, but is not limited to, a first data source 502A, a second data source 502B, and a third data source 502C.

In an embodiment, the first data source 502A may correspond to legacy medical knowledge. Specifically, the legacy medical knowledge refers to traditional or historical medical practices, theories, and information that have been passed down through generations and may encompass the methods, beliefs, and treatments used in medicine. In an embodiment, the legacy medical knowledge may include textbooks associated with the medical field, research papers associated with the medical field, patents associated with the medical field, publications associated with the medical field, clinical trials, and research databases, healthcare guidelines, and protocols, medical conferences and symposia, drug databases and formularies, public health reports and epidemiological data, and the like.

In an embodiment, the second data source 502B and the third data source 502C may be associated with at least one patient 504 (of multiple patients). The second data source 502B may correspond to medical data that may be obtained from the set of medical devices 112 that may be associated with the patient 504 to capture corresponding parameters associated with the patient 504. In an embodiment, the third data source 502C may be associated with the patient 504 (of multiple patients) and may correspond to medical data that may be obtained from the set of scanning devices 114.

In an embodiment, each of the set of scanning devices 114 may transmit the captured first medical data to the one or more storage servers 504. In an embodiment, the one or more storage servers may correspond to a Picture Archiving and Communication System (PACS) server. The set of MR databases 106 may be configured to transmit one or more API calls to the one or more storage servers 504 for the retrieval of the first medical data captured by the set of scanning devices 114. Once retrieved, the first medical data may be stored in the set of MR databases 106. In an embodiment, the one or more API calls may correspond to Fast Healthcare Interoperability Resources (FHIR) API calls. The system 102 may be further configured to retrieve the first medical data from the set of MR databases 106, for example, using the FHIR API calls

In an embodiment, metadata associated with the first patient may be embedded within the first medical data in the set of MR databases 106. Such metadata may correspond to the medical data that may be associated with the first patient. By way of example and not limitation, such metadata may include, but is not limited to, doctor's notes, lab reports, and the like. The system 102 may receive the first medical data along with the metadata from the set of MR databases 106. In an embodiment, the doctor's notes may not be scanned and may be retrieved via the FHIR API call to the set of MR databases 106 where the doctor's note may be stored when dictated by the doctor. For example, if the first medical data corresponds to an X-ray image, then the doctor's note indicative of an analysis of the X-ray image may be stored as the metadata with the first medical data.

In some embodiments, the data obtained from the one or more medical devices may include numerical data. In such instances, the system 102 may be configured to obtain the numerical data captured by one or more medical devices associated with the patient 504. The system 102 may be configured to generate a description associated with the received numerical data and further store the generated description in at least one of the set of MR databases 106.

In an embodiment, the data generated or stored in the set of MR databases 106 may be utilized by the system 102 to train the one or more LLMs 210 to increase the performance of the one or more LLMs 210.

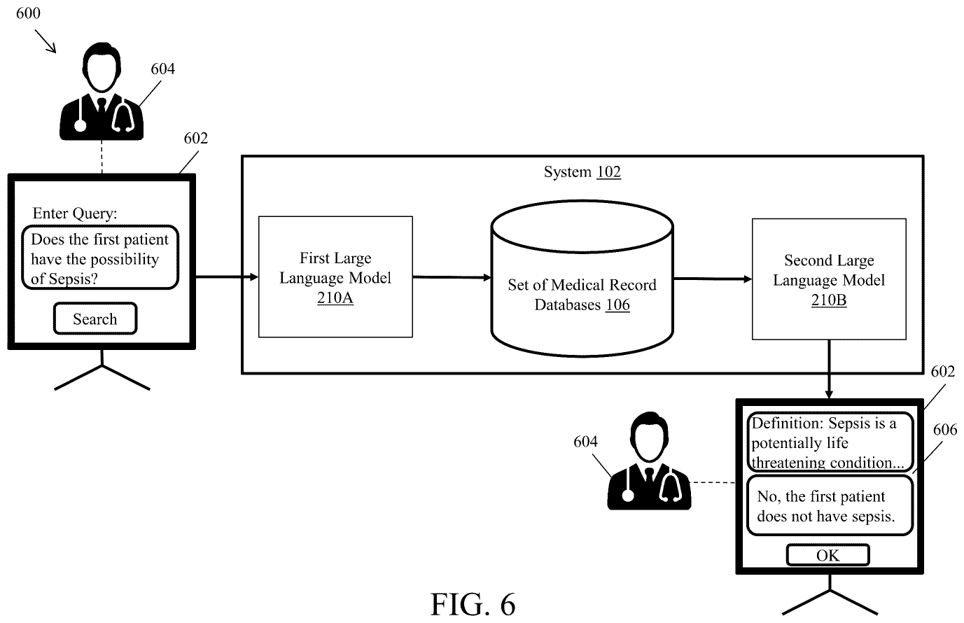

FIG. 6 is a diagram that illustrates an exemplary scenario for an application for medical data governance, in accordance with an embodiment of the disclosure. FIG. 6 is explained in conjunction with elements from FIGS. 1, 2, 3, 4, and 5. With reference to FIG. 6, there is shown an exemplary scenario 600. There is further shown a user device 602 associated with a user 604 who may be a medical professional (for example, a doctor (physician), a nurse, a surgeon, a pharmacist, a dentist, a therapist, and the like).

In an embodiment, the user 604 may wish to determine information related to medical field or related to a patient (say the first patient 116) whom the user 604 may be treating. The user 604 may enter a query related to the medical field or related to a patient (say the patient 504) whom the user 604 may be treating on the user device 602. Based on the reception of the query, the user device 602 may transmit the query to the system 102 via the communication network 110. By way of a first example and not limitation, the query may be “Does the first patient have the possibility of Sepsis?”.

Based on the reception of the query from the user device 602, the system 102 may be configured to apply a first LLM 210A of the one or more LLMs 210 on the received at least one search query. Based on the application of the one or more LLMs 210, the system 102 may determine metadata associated with the at least one search query. The one or more LLMs 210 may be pre-trained to determine metadata associated with the search query. With reference to the first example, the one or more LLMs may find what is the metadata needed to determine whether there is any possibility of the first patient 116 suffering from sepsis.

The system 102 may be further configured to query the first MR database 106A of the set of MR databases 106 based on the determined metadata to retrieve output data. The output data may be associated with the search query and may include at least the possibility of the first patient 116 suffering from sepsis.

In an embodiment, the system 102 may be configured to extract raw data associated with the determined metadata from the first MR database 106A. The system 102 may be further configured to apply the one or more LLMs 210 on the extracted raw data. Specifically, the system 102 may be configured to apply a second LLM 210B of the one or more LLMs 210 on the extracted raw data. Based on the application of the one or more LLMs 210 on the extracted raw data, the system 102 may be configured to retrieve the output data. The output data may correspond to an answer to the query. The system 102 may be further configured to transmit the retrieved first medical data to the user device 602. The user device 602 may receive the output data and render the first medical data on a user interface (UI) of the user device 606.

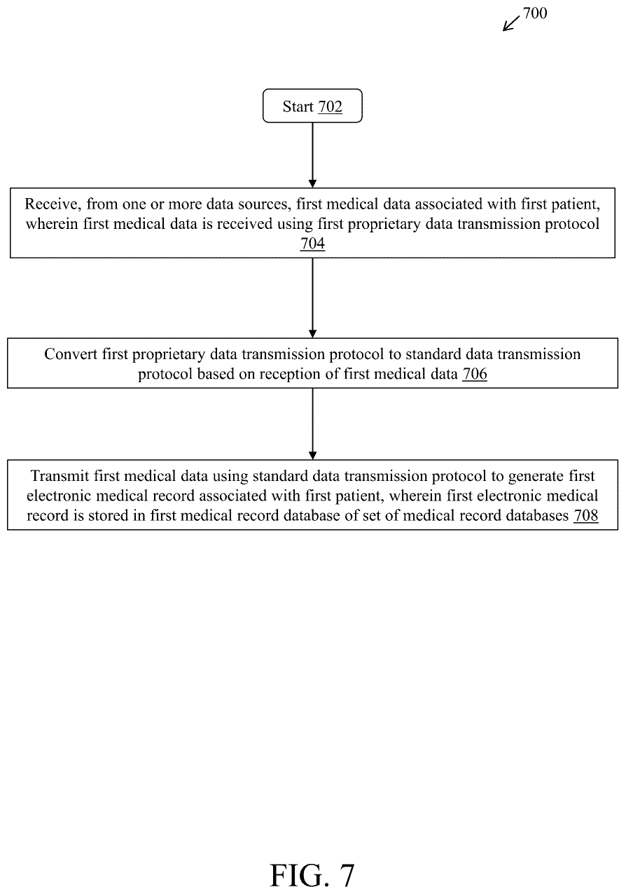

FIG. 7 is a flowchart that illustrates an exemplary method for data transfer mechanism between medical devices for medical data governance, in accordance with an embodiment of the disclosure. FIG. 7 is explained in conjunction with elements from FIGS. 1, 2, 3, 4, 5, and 6. With reference to FIG. 7, there is shown a flowchart 700. The operations of the exemplary method may be executed by any computing system, for example, by the system 102 of FIG. 1 or the circuitry 202 of FIG. 2. The operations of the flowchart 700 may start at 702 and may proceed to 704.

At 704, the first medical data associated with the first patient 116 may be received from one or more data sources 104. The first medical data may be received using a first proprietary data transmission protocol. In at least one embodiment, the circuitry 202 may receive, from the one or more data sources 104, the first medical data associated with the first patient 116, wherein the first medical data is received using a first proprietary data transmission protocol.

At 706, the first proprietary data transmission protocol may be converted to a standard data transmission protocol based on the reception of the first medical data. In at least one embodiment, the circuitry 202 may convert the first proprietary data transmission protocol to the standard data transmission protocol based on the reception of the first medical data.

At 708, the first medical data may be transmitted using the standard data transmission protocol to generate a first electronic medical record associated with the first patient 116. The first electronic medical record may be stored in the first medical record database 106A of a set of medical record databases 106. In at least one embodiment, the circuitry 202 may transmit the first medical data using the standard data transmission protocol to generate the first electronic medical record associated with the first patient 116, wherein the first electronic medical record is stored in the first medical record database 106A of the set of medical record databases 106.

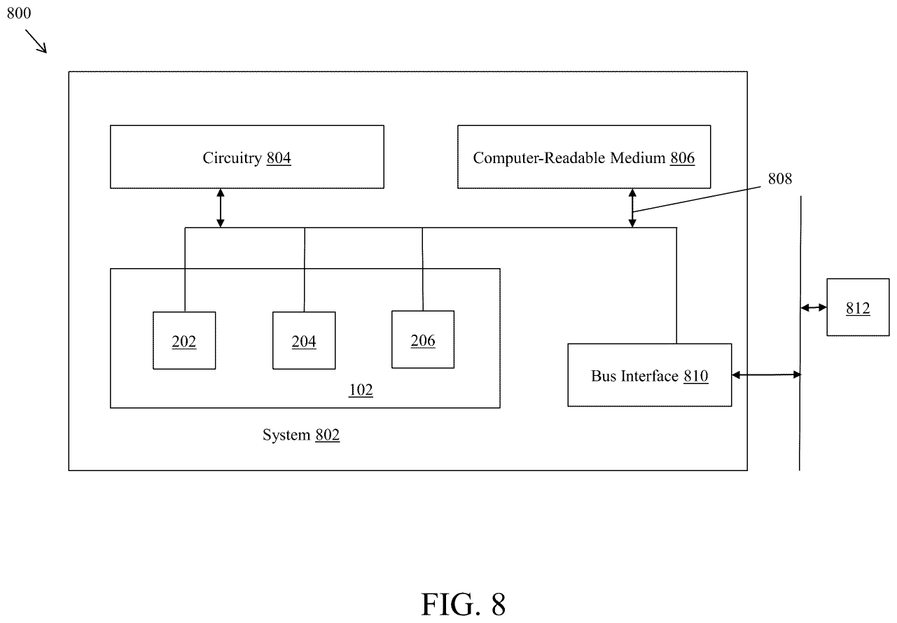

FIG. 8 is a conceptual diagram illustrating an example of a hardware implementation for a system used for data transfer mechanism between medical devices for medical data governance, in accordance with an exemplary embodiment of the disclosure. FIG. 8 is explained in conjunction with elements from FIGS. 1, 2, 3, 4, 5, 6, and 7. Referring to FIG. 8, the hardware implementation shown by a representation 800 for the network environment 100 employs a processing system 802 for data transfer mechanism between medical devices for medical data governance, in accordance with an exemplary embodiment of the disclosure, as described herein.

In some examples, the processing system 802 may comprise a circuitry 804, a non-transitory computer-readable medium 806, a bus 808, a bus interface 810, and a transceiver 812.

The circuitry 804, such as the circuitry 202, may be configured to manage the bus 808 and general processing, including the execution of a set of instructions stored on the non-transitory computer-readable medium 806. The set of instructions, when executed by the circuitry 804, causes the system 102 to execute the various functions described herein for any particular apparatus. The circuitry 804 may be implemented, based on a number of processor technologies known in the art. Examples of the circuitry 804 may be RISC processor, ASIC processor, CISC processor, and/or other processors or control circuits.

The non-transitory computer-readable medium 806 may be used for storing data that is manipulated by the circuitry 804 when executing the set of instructions. The data is stored for short periods or in the presence of power.

The bus 808 may be configured to link together various circuits. In this example, the network environment 100 employing the processing system 802 and the non-transitory computer-readable medium 806 may be implemented with bus architecture, represented generally by bus 808. The bus 808 may include any number of interconnecting buses and bridges depending on the specific implementation of the system 102 and the overall design constraints. The bus interface 810 may be configured to provide an interface between the bus 808 and other circuits, such as the transceiver 812, and external devices, such as the display device 108A, and the server 108.

The transceiver 812 may be configured to provide a communication of the system 102 with various other apparatus, such as the display device 108A, via a network. The transceiver 812 may communicate via wireless communication with networks, such as the Internet, the Intranet and/or a wireless network, such as a cellular telephone network, a wireless local area network (WLAN) and/or a metropolitan area network (MAN). The wireless communication may use any of a plurality of communication standards, protocols and technologies, such as 5th generation mobile network, Global System for Mobile Communications (GSM), Enhanced Data GSM Environment (EDGE), Long Term Evolution (LTE), wideband code division multiple access (W-CDMA), code division multiple access (CDMA), time division multiple access (TDMA), Bluetooth, Wireless Fidelity (Wi-Fi) (such as IEEE 802.11a, IEEE 802.11b, IEEE 802.11g and/or IEEE 802.11n), voice over Internet Protocol (VoIP), and/or Wi-MAX.

It should be recognized that, in some embodiments of the disclosure, one or more components of FIG. 8 may include software whose corresponding code may be executed by at least one processor, for across multiple processing environments.

In an aspect of the disclosure, the circuitry 804, the non-transitory computer-readable medium 806, or a combination of both may be configured or otherwise specially programmed to execute the operations or functionality of the circuitry 202, the memory 204, the I/O device 206, the network interface 208, and the one or more LLMs 210 or various other components described herein, as described with respect to FIGS. 1 to 8.

Various embodiments of the disclosure comprise the system 102 for data transfer mechanism between medical devices for medical data governance. The system 102 may comprise, for example, the circuitry 202, the memory 204, the I/O device 206, and the network interface 208, and/or the one or more LLMs 210. The circuitry 202 of the system 102 may be configured to receive, from the one or more data sources 104, first medical data associated with the first patient 116. The first medical data may be received using the first proprietary data transmission protocol. The circuitry 202 may be further configured to convert the first proprietary data transmission protocol to the standard data transmission protocol based on the reception of the first medical data. The circuitry 202 of the system 102 may be further configured to transmit the first medical data using the standard data transmission protocol to generate the first electronic medical record associated with the first patient 116. The first electronic medical record is stored in a first medical record database 106A of the set of medical record databases 106.

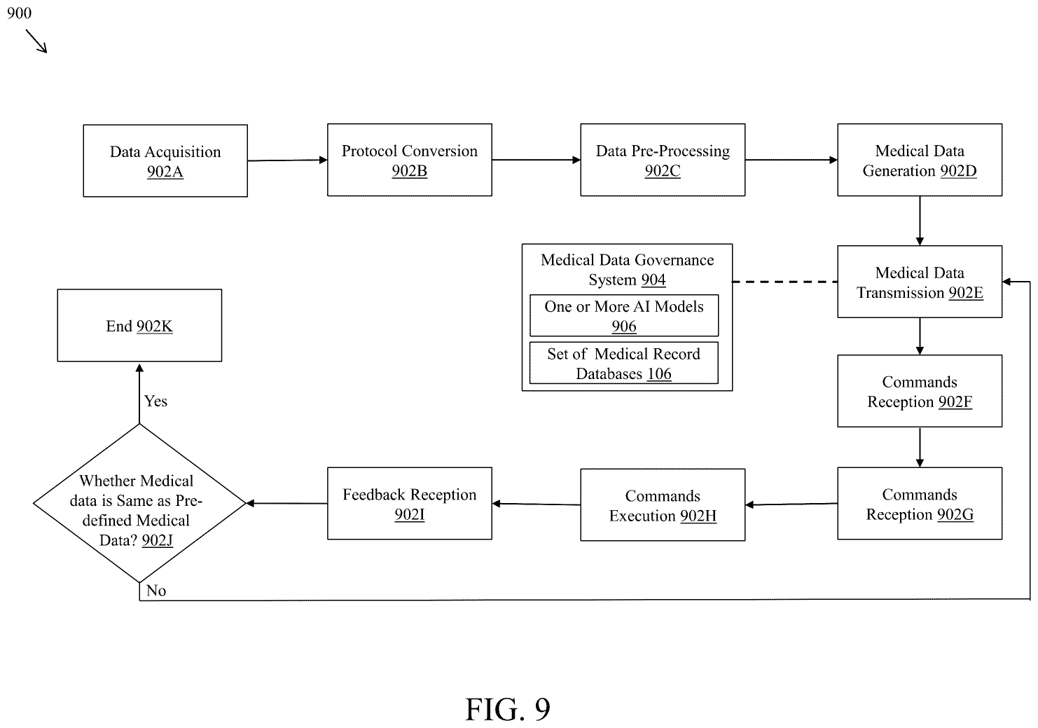

FIG. 9 is a diagram that illustrates exemplary operations for execution of a set of commands, in accordance with an embodiment of the disclosure. FIG. 9 is explained in conjunction with elements from FIG. 1, FIG. 2, FIG. 3, FIG. 4, FIG. 5, FIG. 6, FIG. 7, and FIG. 8. With reference to FIG. 9, there is shown a block diagram 900 that illustrates exemplary operations from 902A to 902K, as described herein. The exemplary operations illustrated in the block diagram 900 may start at 902A and may be performed by any computing system, apparatus, or device, such as by the system 102 of FIG. 1 or circuitry 202 of FIG. 2. Although illustrated with discrete blocks, the exemplary operations associated with one or more blocks of the block diagram 900 may be divided into additional blocks, combined into fewer blocks, or eliminated, depending on the particular implementation.

In an embodiment, the system 102 may operate in two phases—a setup phase and an execution phase. In the setup phase, the system 102 may be initialized to perform the one or more operations in the execution phase. In the set-up phase, the system 102 may be produced physically at an industry or a factory. During the production of the system 102, the system 102 is produced with all communication components active by a foundry or assembly subcontractor, based on an Open Systems Interconnection (OSSI) design. During Just-in-Time (JIT) production, the system 102 may be customized in such a manner that only the connectors relevant to the set of medical devices 112 are exposed (e.g., USB, RS232, proprietary interfaces) and the system's 102 software is tailored for a set of medical devices 112 (e.g., drivers, protocol conversions, security options). Unused connectors remain unexposed, reducing risks of unintended use, configuration errors, or operational risks. The JIT approach ensures that only two types of motherboards are produced, both with identical external connectors but differing internal circuitry. These motherboards can be configured for any supported medical device, facilitating stock provisioning and delivery while enabling device-specific customization.

After the production of the system 102 at the industry or the factory, the system 102 arrives at the healthcare facility with the appropriate hardware interfaces and pre-installed device-specific software. It is physically attached to one or more data sources 104, often sharing the power source (e.g., PoE, USB, or device's 12V output). The primary RJ45 port handles both data and power options (PoE, USB power, or device-supplied power) whereas secondary or tertiary RJ45 ports may connect to other devices or provide pass-through connections. In an embodiment, additional ports (USB, RS232, IR, wireless) are present as needed, depending on JIT configurations. Furthermore, the system 102 may be powered simultaneously by PoE, USB, or direct device power. This redundancy reduces the risk of communication or operational failures due to power interruptions. Once powered, the system 102 automatically recognizes the attached medical device type (e.g., ventilator, infusion pump) via its specific drivers and configuration.

After the physical connection and power integration to the system 102, an initialization of the system 102 may be required. Once the system 102 boots up, the circuitry 202 and firmware of the system 102 may initialize its communication interfaces. After initialization of the communication interfaces, a handshake may occur with a Medical Data Governance (MDG) system. Such handshake may register Device Type (e.g., “Infusion Pump”), Location (e.g., “ICU Bed #3”), and Capabilities (e.g., “Can set alarm thresholds”). After the handshake with the MDG system, the system 102 may connect to a time source (NTP server, GPS, or local reference clock) to establish a precise internal clock such that all subsequent data is accurately timestamped upon arrival from the medical devices. Furthermore, if a contextual feedback device in the same patient area is present, the contextual Feedback device discovers the system 102, setting up a local data exchange path for error detection, redundancy, and metadata generation. After this the set-up phase may be completed and the system 202 may be ready to operate in the execution phase. In the execution phase, the system 102 may perform operations as described below from 902A to 902K.

At 902A, a data acquisition operation may be performed. In data acquisition operation, the circuitry 202 may be configured to receive the first medical data. The first medical data may be associated with the first patient 116. In an embodiment, the first patient 116 may be suffering from a medical condition (say a disease) and may be admitted to a hospital. The set of medical devices 112 may be configured to capture the medical data associated with the first patient 116. In an embodiment, the set of medical devices 112 may further include a set of diagnostic medical devices and a set of monitoring devices.

Each of the set of diagnostic medical devices may correspond to instruments or tools that may be used by healthcare professionals to identify and diagnose the medical conditions in the first patient 116. Each of the set of diagnostic medical devices may often provide quantitative or qualitative data about the health status of the first patient 116. Such data may be useful in determining an appropriate course of treatment for the medical condition. In an embodiment, the set of diagnostic medical devices may include, but are not limited to, a blood glucose monitor, an electrocardiogram (ECG or EKG) machine, a spirometer, and a sphygmomanometer.

Each of the set of monitoring devices may correspond to instruments that may be designed to observe and track various physiological parameters or medical conditions in the first patient 116. Each of the set of monitoring devices may provide real-time data, enabling healthcare professionals to make informed decisions about patient care and treatment adjustments. Examples of such monitoring devices may include, but are not limited to, a pulse oximeter, a glucose monitor, a Holter monitor, a blood pressure monitor, a cardiac monitor, a respiratory rate monitor, a sleep pane monitor, an intracranial pressure monitor, and a wearable fitness tracker.

In an embodiment, the first medical data may be received from one or more devices of the set of scanning devices. Each of the set of scanning devices may correspond to equipment that may be used in healthcare settings to obtain detailed images or scans of internal structures within the human body for diagnostic or monitoring purposes. Examples of the set of scanning devices may include, but are not limited to, an X-ray machine, an MRI (Magnetic Resonance Imaging) scanner, a CT (Computed Tomography) scanner, an ultrasound machine, a Positron Emission Tomography (PET) Scanner, a Single-Photon Emission Computed Tomography (SPECT) Scanner, a Mammography Machine, and an ultrasound machine.

In an embodiment, the system 102 may be configured to receive the first medical data associated with the first patient 116 from the first medical device 112A of the set of medical devices 112 or the first scanning device 114A of the set of scanning devices 114. As discussed above, both the set of medical devices 112 and the set of scanning devices 114 may be included in the one or more data sources 104. In an embodiment, the first medical data may correspond to at least one of a medical professional's prescription note, a pathology report, an X-radiation (X-RAY) report, a computed tomography (CT) report, a magnetic resonance imaging (MRI) report, an ultrasound report, a cardiac catheter report, or a cardiac stress report associated with the first patient 116.

In an embodiment, the first medical device 112A (or the first scanning device 114A) may use the first proprietary data transmission protocol to transmit the data from the first medical device 112A (or the first scanning device 114A) to the system 102. Therefore, the system 102 may receive the first medical data using the first proprietary data transmission protocol.

In an embodiment, the system 102 may be configured to receive the first medical data from one or more storage servers associated with the one or more data sources 104. In such an implementation, the system 102 may be configured to receive the first medical data using one or more application programming interface (API) calls. By way of example and not limitation, each of the set of scanning devices 114 may be configured to transmit the first medical data (that may include images (say X-ray images)) to the one or more storage servers. In an implementation, the one or more storage servers may correspond to a Picture Archiving and Communication System (PACS) server. Further, the system 102 may transmit a Fast Healthcare Interoperability Resources (FHIR) API call to the PACS server. Based on the reception of the FHIR API call, the PACS server may transmit the first medical data to the system 102.

In another embodiment, the first medical data stored in the PACS server may be transmitted to the set of MR databases 106 using the FHIR API call. In the set of MR databases, metadata associated with the first patient may be embedded within the first medical data. Such metadata may correspond to the medical data that may be associated with the first patient. In an embodiment, the medical data may have been captured by the set of medical devices 112 or the set of scanning devices 114. By way of example and not limitation, such metadata may include, but is not limited to, doctor's notes, lab reports, and the like. The system 102 may receive the first medical data along with the metadata from the set of MR databases 106. In an embodiment, the doctor's notes may not be scanned and may be retrieved via the FHIR API call to the set of MR databases 106 where the doctor's note may be stored when dictated by the doctor.

In an alternate embodiment, the system 102 may be configured to receive the first medical data from the set of medical devices 112 and the set of scanning devices 114 wirelessly. In such implementation, each of the set of medical devices 112 and the set of scanning devices 114 may be configured to wirelessly transmit the captured first medical data to the system 102.

In an embodiment, the set of medical devices 112 may transmit the raw data (or the first medical data) such as, but not limited to, vital signs, alarm statuses, infusion rates, etc. via its native protocol (e.g., serial, USB, IR, or proprietary messages). For example, the set of medical devices 112 may transmit the first medical data (e.g., heart rate=80 bpm, blood pressure=120/80, ventilator FiO2=0.4) via its native protocol to the system 102.

At 902B, a protocol conversion operation may be executed. In the protocol conversion operation, the system 102 may be configured to convert the first proprietary data transmission protocol to a standard data transmission protocol. In an embodiment, the standard data transmission protocol may be used by the set of medical record databases 106.

In an embodiment, the first medical data may have to be transferred to the other medical devices (say the second medical device 112B) that may use a second proprietary data transmission protocol. In such an embodiment, the system 102 may be further configured to convert the standard data transmission protocol to the second proprietary data transmission protocol before transmission.

In an embodiment, the system 102 may be configured to parse the first proprietary data transmission protocol based on the received first medical data. Based on the parsing, the system 102 may be configured to convert the first proprietary data transmission protocol to the standard data transmission protocol. Specifically, the system 202 may parse the first proprietary data transmission protocol (or the native protocol) and translate it into the standard data transmission protocol suitable for downstream systems. Examples of the standard data transmission protocol may include, but are not limited, to a Health Level 7 (HL7) protocol, a Fast Healthcare Interoperability Resources (FHIR) protocol, a JavaScript Object Notation (JSON) protocol, an extensible Markup Language (XML) protocol, a Clinical Document Template (CDT) protocol, and an Open-Source Software Initiative's Full Disclosure Protocol (OSSI's Full Disclosure Protocol).

In an embodiment, the HL7 and FHIR protocols may be primarily used because they are the most widely recognized and adopted standards for medical data exchange. The HL7 is the most commonly used protocol for streaming continuous medical data due to its simplicity and effectiveness in real-time communication scenarios. On the other hand, the FHIR (Fast Healthcare Interoperability Resources) is a new and promising protocol that leverages the REST architecture to provide modern, resource-based APIs. FHIR enables faster, modular, and scalable integrations, making it an ideal choice for the evolving needs of healthcare interoperability.

The CDT protocol may be ideal for unsolicited continuous data streaming due to its simplicity and low overhead. The JSON and XML protocols may be particularly suited for REST-based implementations. The OSSI also implements a proprietary protocol designed to support robust and complex data communication. This protocol enables real-time remote display of data without interfering with third-party systems utilizing HL7, FHIR, CDT, JSON, or XML. It ensures OSSI-specific applications and systems can operate independently while maintaining seamless interoperability with external systems. The HL7 protocol may be best suited for streaming continuous medical data, particularly in scenarios requiring unsolicited, real-time data delivery to receivers. The FHIR protocol may be built on REST and may be highly effective for modular and resource-based integrations, allowing lightweight, secure, and scalable communication. The CDT protocol may be a simple and efficient choice for continuous data streaming in legacy or straightforward systems. The OSSI's proprietary protocol offers advanced capabilities for real-time visualization and interaction, enhancing OSSI-specific applications while remaining non-disruptive to other data consumers and the REST-based Integrations (JSON, XML, FHIR) may be ideal for APIs, enabling interaction with systems that rely on modem web standards. Details about REST are known in the art and therefore are not provided for the sake of brevity.

In an embodiment, a protocol conversion module of the system 202 may be implemented as a System on a Module (SOM) electronic circuit that may be designed to ensure seamless communication between medical devices and the MDG system. The protocol conversion module may be compact, versatile, and built with uniformity in mind, featuring at least three standardized RJ45 connectors while accommodating diverse medical devices and may be responsible for the conversion of the protocols.

At 902C, a data pre-processing operation may be executed. In the data pre-processing operation, the system 102 may be configured to apply one or more pre-processing operations on the first medical data based on the conversion. In an embodiment, the application of the one or more pre-processing operations on the first medical data corresponds to appending a timestamp to the first medical data. Specifically, each data message is assigned a precise timestamp, ensuring all device outputs across the hospital are synchronized for an accurate correlation.

In an embodiment, a time synchronization module of the system 102 may be configured to append the timestamp to the first medical data. The time synchronization module may be one of the most critical functionalities in medicine, addressing the pervasive issue of medical devices often having inaccurate or incorrect time references. The accuracy of time is fundamental for effective medical analysis, enabling healthcare professionals and AI systems to make reliable cause-and-effect evaluations and ensure optimal patient outcomes. The primary objective of the time synchronization module may be to assign precise and unified timestamps to data collected from diverse medical devices, regardless of their internal clock accuracy. Furthermore, the time synchronization module may be configured to synchronize data streams from multiple devices, enabling the seamless integration of measurements for clinical decision-making. The time synchronization module may provide the temporal precision necessary for one or more artificial intelligence (AI) models 906 and medical experts to analyze data in real-time or retrospectively, and to eliminate errors introduced by mismatched or unreliable time references across different devices.

In an embodiment, the time synchronization module uses a highly accurate time source, such as Network Time Protocol (NTP) servers, Global Positioning System (GPS), amps, or a local, precision timekeeping device (e.g., an atomic clock or high-precision oscillator). This centralized source ensures all connected devices adhere to a unified, precise time standard.

As data is transmitted from a medical device of the set of medical devices 112, the time synchronization module immediately assigns a timestamp based on the centralized time source. The time synchronization module ensures that every piece of information has a consistent and accurate time reference, regardless of the medical device's internal clock. The time synchronization module synchronizes time across all connected devices, aligning their data streams. This is particularly important when multiple devices monitor the same patient or event (e.g., heart rate monitor, blood pressure cuff, drug infusion pump). In an embodiment, for scenarios requiring extreme accuracy, the time synchronization module provides sub-second synchronization, essential for capturing events occurring in rapid succession (e.g., drug effects or medical interventions).

As an example, a blood pressure measurement must be timestamped accurately to correlate with other physiological data, such as heart rate or oxygen saturation. If the timestamp is inaccurate, even by a few seconds, it could lead to incorrect interpretations of a patient's condition. Furthermore, understanding whether a drug's effect preceded or succeeded its administration is vital for assessing efficacy and safety. For example, an infusion pump may administer a drug at 10:15:30.500 (hours:minutes:seconds.milliseconds), a patient monitor detects a physiological change (e.g., blood pressure drops) at 10:15:31.200. In such a case, accurate timestamps confirm that the effect followed the administration, providing critical insights into the drug's impact.

In emergencies, precise timing of interventions like defibrillation, CPR, or drug administration can mean the difference between life and death. Therefore, accurate timestamps ensure that the sequence of events is clear and actionable. Also, the one or more AI models analyzing patient data rely on precise timestamps to identify patterns and correlations. For example, detecting subtle correlations between drug administration and cardiac events could require synchronization across multiple devices.

By synchronizing all device data to a unified clock, the system 102 may accurately align events such as drug administration times, patient monitor readings, and changes in ventilator settings. This ensures a cause-and-effect analysis can be performed reliably (e.g., “Did the blood pressure change occur before or after administering a medication?”). In advanced applications, sub-second precision helps AI models correlate fast physiological changes.

Therefore, it may be noted that time synchronization is important for improved accuracy in diagnosis because it eliminates ambiguities caused by mismatched device clocks, ensuring that medical professionals and AI systems have reliable data for decision-making. Time synchronization is important for enhanced patient safety as time synchronization accurately correlates events, such as drug administration and adverse effects, reducing risks and improving patient outcomes. Furthermore, time synchronization may be important in compliance with standards because it ensures adherence to medical standards and protocols that often require precise time documentation for audits and legal purposes. Also, time synchronization may be important in cross-device integration because it allows seamless integration of data from multiple devices, forming a cohesive picture of the patient's condition.

At 902D, a medical data generation operation may be executed. In the data generation operation, the system 102 may be configured to generate second medical data based on the application of one or more pre-processing operations on the first medical data. In an embodiment, the system 102 may be configured to standardize the first medical data and append the timestamp to the first medical data to generate the second medical data. In an embodiment, the standardized and timestamped first medical data may correspond to the second medical data associated with the first patient.

At 902E, a medical data transmission operation may be executed. In the medical data transmission operation, the system 202 may be configured to transmit the second medical data, using the standard data transmission protocol, to a medical data governance (MDG) system 904. The MDG system 904 may implement the MDG and may include the set of medical record databases 106 and one or more AI models 906. In an embodiment, the MDG system may be hosted on a server external to system 102.

Each AI model of one or more AI models 906 may be a computational network or a system of artificial neurons, arranged in a plurality of layers, as nodes. The plurality of layers of each AI model of one or more AI models 906 may include an input layer, one or more hidden layers, and an output layer. Each layer of the plurality of layers may include one or more nodes (or artificial neurons). Outputs of all nodes in the input layer may be coupled to at least one node of hidden layer(s). Similarly, inputs of each hidden layer may be coupled to outputs of at least one node in other layers of each AI model of one or more AI models 906. Outputs of each hidden layer may be coupled to inputs of at least one node in other layers of each AI model of one or more AI models 906. Node(s) in the final layer may receive inputs from at least one hidden layer to output a result. The number of layers and the number of nodes in each layer may be determined from the hyper-parameters of the neural network 112. Such hyper-parameters may be set before or while training each AI model of one or more AI models 906 on a training dataset.

Each node of each AI model of one or more AI models 906 may correspond to a mathematical function (e.g., a sigmoid function or a rectified linear unit) with a set of parameters, tunable during training of the network. The set of parameters may include, for example, a weight parameter, a regularization parameter, and the like. Each node may use the mathematical function to compute an output based on one or more inputs from nodes in other layer(s) (e.g., previous layer(s)) of each AI model of one or more AI models 906. All or some of the nodes of each AI model of one or more AI models 906 may correspond to the same or a different mathematical function.

In training of each AI model of one or more AI models 906, one or more parameters of each node of each AI model of one or more AI models 906 may be updated based on whether an output of the final layer for a given input (from the training dataset) matches a correct result based on a loss function for each AI model of one or more AI models 906. The above process may be repeated for the same or a different input until a minima of loss function may be achieved and a training error may be minimized. Several methods for training are known in art, for example, gradient descent, stochastic gradient descent, batch gradient descent, gradient boost, meta-heuristics, and the like.

Each AI model of one or more AI models 906 may include electronic data, such as, for example, a software program, code of the software program, libraries, applications, scripts, or other logic or instructions for execution by a processing device, such as circuitry. Each AI model of one or more AI models 906 includes code and routines configured to enable a computing device, such as the electronic device 102 to perform one or more operations. Additionally, or alternatively, each AI model of one or more AI models 906 may be implemented using hardware including a processor, a microprocessor (e.g., to perform or control the performance of one or more operations), a field-programmable gate array (FPGA), or an application-specific integrated circuit (ASIC). Alternatively, in some embodiments, each AI model of one or more AI models 906 may be implemented using a combination of hardware and software. Although in FIG. 1, the neural network 112 is shown integrated within the electronic device 102, the disclosure is not so limited. Accordingly, in some embodiments, each AI model of one or more AI models 906 may be a separate entity in the electronic device 102, without deviation from scope of the disclosure. In an embodiment, each AI model of one or more AI models 906 may be stored in a server. Examples of each AI model of one or more AI models 906 may include, but are not limited to, a deep neural network (DNN), a convolutional neural network (CNN), a CNN-recurrent neural network (CNN-RNN), R-CNN, Fast R-CNN, Faster R-CNN, an artificial neural network (ANN), (You Only Look Once) YOLO network, a fully connected neural network, and/or a combination of such networks.

In an embodiment, the second medical data is transmitted to the MDG system 904 through the network interface 208. Meanwhile, full raw data via OSSI protocol can be stored or archived for audit trails and future reference. The MDG system 904 (or connected AI services) receives real-time, standardized data from the system 102. The system 102 may control the MDG system 904 to apply the one or more AI models 906 on the second medical data. The system 102 may further control the MDG system 904 to generate a first set of commands based on the application of the one or more AI models 906 on the second medical data.

In an embodiment, if an AI model of one or more AI models 906 identifies a risk (say possible sepsis), it suggests corrective actions (or the first set of commands). The MDG system 904, guided by clinical protocols or direct physician oversight, may formulate the first set of commands (e.g., “Increase IV fluids”) based on the corrective actions and send it back through the system 202. In an embodiment, a doctor or the authorized one or more AI models in the MDG system 904 determines a need to adjust a medical device parameter (e.g., reduce ventilator pressure, or change alarm thresholds). The first set of commands is formulated in a standard format (e.g., HL7 order message, FHIR operation).

At 902F, a commands reception operation may be executed. In the commands reception operation, the system 102 may be configured to receive the first set of commands from the MDG system 904 based on the transmitted second medical data. As discussed above, the first set of commands may be generated by the one or more AI models 906. Specifically, the first set of commands may be outputted by the one or more AI models 906 when the second medical data is provided as input to the one or more AI models 906.

Each command of the first set of commands may represent critical, contextual actions that must be executed remotely by the system 202 on a medical device. These contextual actions have the potential to significantly impact the outcomes of the first patient, necessitating the highest levels of security, validation, and contextualization. This approach seeks to address one of the major limitations in current medical device management i.e., the inability to perform certain remote actions due to safety and risk concerns.

Today, certain medical actions require the physical presence of a doctor with the patient and medical device. This (i.e., remote generation of commands) ensures that the doctor can observe all relevant information, apply their expertise, and validate the appropriateness of the action. However, with advancements in remote monitoring and decision support systems, the contextual information and rationale for these decisions can now be digitally collected, presented, and validated, enabling safe and effective remote control of medical devices.

Therefore, the system 102 ensures contextualized decision-making as the doctor must be presented with all relevant patient data (e.g., vitals, lab results, device outputs) in an integrated view to make an informed decision. This contextual information must be validated, ensuring the data is complete, accurate, and up-to-date before any command can be issued remotely. The system 102 further ensures safety and error prevention as automated safety checks by the system 102 prevent obvious errors (e.g., setting alarm thresholds outside safe limits or administering drugs at harmful dosages). The system 102 incorporates decision-support rules, such as ensuring ventilator settings are appropriate for the patient's current oxygenation levels and respiratory condition.

At 902G, a commands validation operation may be executed. In the commands validation operation, the system 102 may be configured to validate the first set of commands. For example, in case of drug administration, if the command is to administer a specific dose of a drug via an infusion pump, then the system 102 may validate the command by ensuring the dosage is within the safe range for the patient's weight, age, and condition and confirming there are no known contraindications or active drug interactions. As another example in case of ventilator adjustments, if the command is to increase or decrease pressure or oxygen delivery in a ventilator, then the system 102 may validate the command by ensuring the adjustment aligns with current blood gas readings and oxygen saturation levels and confirming that recent changes to other devices (e.g., oxygen flow meters) do not conflict with the ventilator settings. As another example in case of alarm configuration if the command is to adjust alarm thresholds for heart rate, blood pressure, or oxygen levels, then the system 102 may validate the command by ensuring the thresholds are realistic and patient-specific and verifying active monitoring is ongoing to avoid false positives or undetected critical events. As another example in case of a defibrillation preparation if the command is to prime and prepare a defibrillator for use during cardiac arrest, then the system 102 may validate the command by confirming the patient's current condition warrants defibrillation based on the ECG readings and ensuring proper energy levels are selected to match the patient's age and cardiac profile. As another example in case of a fluid balancing if the command is to adjust IV pump rates for fluid administration or diuresis, then the system 102 may validate the command by verifying current fluid balance, electrolyte levels, and renal function to prevent overload or dehydration and cross-checking with weight and recent fluid input/output measurements.

In an embodiment, the system 102 may employ a validation framework to validate each command of the first set of commands. The validation framework may validate each command of the first set of commands based on one or more criteria such as, but not limited to, a source of the commands, contextual validation, and a feedback loop. In an embodiment, the validation framework may ensure that the first set of commands must originate from qualified personnel (e.g., a doctor) and be issued through secure, authenticated channels. The system 102 records the contextual information presented to the doctor, ensuring that their rationale for the command is traceable. Further the system 102 integrates data from multiple sources (e.g., patient monitors, lab systems, historical records) to provide a complete picture. The system 102 runs safety checks, comparing the command against established guidelines and patient-specific parameters. After the command is executed, the system 102 monitors the medical device's response and the patient's condition to confirm the desired effect. If discrepancies are detected (e.g., the ventilator did not achieve the expected pressure), alerts are generated for immediate intervention.

In an embodiment, the key objective for the validation of the first set of commands may be to enhance safety by preventing errors by validating commands against patient-specific data and safety guidelines. Other key objectives may be to enable remote care that allows doctors to perform high-impact actions remotely without compromising patient safety. Another key objective may be to ensure accountability by recording the contextual information presented to the decision-maker, ensuring traceability and accountability. Another key objective may be to support AI-assisted decisions by using the AI models to flag potential errors or provide recommendations based on patient data trends.

Examples of safety check analogies may be drug administration validation that is widely used in healthcare, ensuring drug dosages align with patient weight, age, and safety thresholds, diabetes management as insulin pump systems cross-check blood glucose levels with the administered insulin dosage to prevent hypoglycemia or hyperglycemia and surgical robots as advanced systems validate commands against pre-programmed safety parameters to prevent inadvertent harm during remote-controlled procedures.

At 902H, a command execution operation may be executed. In the command execution operation, the system 102 may be configured to control at least one data source to execute the first set of commands. Specifically, the system 102 may be configured to control at least one data source to execute the validated commands from the first set of commands. Specifically, the system 102 translates the standardized command into the native protocol of the medical and sends it to the medical device. The medical device attempts to execute the command of the first set of commands (for e.g., adjusting ventilator pressure).

In an embodiment, protocol conversion ensures that data from diverse medical devices, often using different formats and standards, is normalized into a common format (e.g., HL7, FHIR, JSON). This normalization is essential for enabling two-way communication between devices and the system 202, thereby allowing commands to be issued in the formats that the set of medical devices may interpret. For example, a ventilator receiving a command to adjust pressure levels interprets the message correctly due to protocol conversion, regardless of the original data format of the issuing system.

Furthermore, the protocol conversion ensures that the medical device feedback, such as status updates or error messages, is translated into a format compatible with the system 102. This allows the system 102 to validate whether the command has been executed successfully and whether the intended outcomes have been achieved. For example, if the ventilator is commanded to reduce pressure, the system 102 monitors feedback in a standardized format to confirm the change has occurred. If not, the system generates an alert and raises the generated alert.

At 902I, a feedback reception operation may be executed. In the feedback reception operation, the system 102 may be configured to receive, from the one or more data sources, third medical data associated with the first patient. The second medical data is received after the execution of the first set of commands. In an embodiment, the system 102 receives the third medical data from a contextual feedback device. In an embodiment, the contextual feedback device is a local, independent unit positioned within the environment of the first patient, such as their room or bedside. The primary function of the contextual feedback device is to receive, analyze, and log data from all medical devices associated with the patient, including portable or ambulatory devices that may only provide temporary measurements. This contextual feedback device plays a pivotal role in ensuring data accuracy, consistency, and availability, even under challenging conditions like network or power outages.

In an embodiment, the key functions of the contextual feedback device include error detection and incongruence monitoring. The contextual feedback device independently cross-checks data from multiple sources to identify inconsistencies, errors, or anomalies. For example, if a patient monitor reports a blood pressure reading that significantly deviates from a recent measurement taken by an ambulatory device, the contextual feedback device flags this as incongruent and raises an alert. The contextual feedback device may be configured to identify errors caused by incorrect settings, faulty sensors, or medical device malfunctions. Another key function of the contextual feedback device may be data logging and aggregation. The contextual feedback device collects and logs independent data streams from all connected medical devices, including those that may not have a permanent network connection. For example, portable devices, such as handheld thermometers, glucose monitors, or blood pressure cuffs, often operate intermittently and move between patients. The contextual feedback device ensures these temporary datasets are captured, stored, and correlated with the appropriate patient's records.

Another key function of the contextual feedback device may be metadata generation. By having access to the complete dataset from various medical devices and scanning devices in the patient's vicinity, the contextual feedback device generates additional metadata that would otherwise be unavailable. This metadata includes time-synchronized correlations between different measurements (e.g., the effect of a ventilator adjustment on oxygen saturation) and derived insights based on combined data streams (e.g., trends in vital signs or responses to interventions). Another key function of the contextual feedback device may be local storage for redundancy. In an event of network or power shortages, the device acts as a local storage hub, ensuring no data is lost. This redundancy is crucial for maintaining a complete and uninterrupted dataset for the patient. Once connectivity is restored, the device synchronizes its stored data with the central system.

At 902J, it may be determined whether the third medical data is same as pre-defined medical data. The pre-defined medical data may correspond to an expected medical data after the execution of the first set of commands. If the third medical data is same as the pre-defined medical data, then it may be deemed that the expected results are met and the control may be transferred to the end at 902K.

Otherwise, the control may be transferred back to the 902E where the third medical data may be transferred again to the to the MDG system 904. The system 102 may further control the MDG system 904 to apply the one or more AI models 906 on the second medical data, the third medical data, and the first set of commands. The system 102 may further control the MDG system 904 to generate a second set of commands based on the application of the one or more AI models 906 on the second medical data, the third medical data, and the first set of commands. The system 102 may further receive the generated second set of commands based on the application of the one or more AI models 906 on the first medical data, the one or more commands, and the second medical data. The system 102 may further control at least one data source to execute at least one command of the second set of commands. This loop will execute until the expected results are met.

In an embodiment, the system 102 may be configured to generate audit trails based on the first medical data, the second medical data, and the first set of commands. The system 102 may be further configured to store the generated audit trails. Specifically, every interaction i.e., the medical data received from the one or more data sources 104, the first set of commands sent, the feedback returned, the anomalies detected—is logged by the system 102. The system 102 may further store these logs for audit trails (such as HIPAA, GDPR, etc.). In an embodiment, the full, unaltered raw data stream from the one or more data sources 104 is captured and saved using the OSSI protocol. Such RAW data is invaluable for post-processing, especially if future findings reveal potential interpretation errors or if more advanced AI models need to revisit original signals. In an embodiment, each command is tied to the issuing entity (doctor, nurse, AI subsystem) and includes references to the contextual information that justified the action. This ensures clear responsibility for each medical decision. Also, each command is tied to the issuing entity (doctor, nurse, AI subsystem) and includes references to the contextual information that justified the action. This ensures clear responsibility for each medical decision.

In an embodiment, the system 102 may also be configured to monitor network traffic, block unauthorized or “illegal” data attempts, and report suspicious activities to MDG's security team. Specifically, the contextual feedback device associated with the system 102 may cross-verify signals to detect anomalies, further reducing attack surfaces.

Therefore, the disclosed system ensures safety and reliability as time synchronization eliminates confusion over event sequencing, two-way communication ensures remote commands are executed securely, with immediate feedback, and the OSSI raw data preservation provides a “forensic” record for advanced troubleshooting and future AI re-analysis. Further, the disclosed system provides improved interoperability as legacy and modern devices coexist under one framework, using the protocol conversion module and standardized data formats. Moreover, the two-way control reduces manual workload as two-way control reduces manual workload and real-time feedback loops confirm outcomes, reducing the need for constant bedside checks. Moreover, the disclosed system 102 provides AI-Driven insights provides high-quality, synchronized data that significantly improves AI model accuracy. Also, new or evolving AI models may use the raw data (OSSI protocol) to uncover deeper insights. Further, the disclosed system 102 ensures reduced errors & cybersecurity threats as the security/observer features log all traffic and can block malicious communications. Also, malicious attempts to issue harmful commands can be thwarted at a system level. Moreover, the disclosed system 102 is autonomous and future-ready as the system localized real-time operations and AI integration create a platform for autonomous or semi-autonomous care in the future and local backups ensure resilience in remote or disaster scenarios.

Therefore, the overall flow of the disclosure begins by attaching the protocol conversion module to the one or more data sources 104 and registering it with the MDG system 904. The medical data from the the one or more data sources 104 is converted into standardized formats, time-synchronized, and passed along for predictive analytics or stored locally by the contextual feedback device. Crucially, the full raw data can also be saved via the OSSI protocol, enabling advanced post-processing and future reinterpretation. When necessary, the two-way commands are issued back to the set of medical devices 112 of the one or more data sources 104—validated, executed, and logged for accountability. The localized real-time operations ensure continuous function even without network access, while AI integration uses synchronized data to identify adverse events or recommend interventions. This synergy between protocol conversion, time synchronization, two-way communication, contextual feedback, predictive analytics, and raw data preservation produces a robust, forward-thinking system that enhances patient safety, empowers healthcare providers, and enables future AI-driven innovations.

Also, this disclosure demonstrates strong synergistic interactions among its core components i.e., protocol conversion, time synchronization, predictive analytics, and contextual feedback. These components create a cohesive and dynamic system that significantly enhances medical device interoperability, patient safety, and clinical decision-making. Their interdependence ensures functionalities that cannot be achieved in isolation.

In an embodiment, the synergy between protocol conversion and time synchronization is defined as the protocol conversion module normalizing diverse medical device outputs into standardized formats like HL7, FHIR, JSON, XML, or CDT, ensuring compatibility across devices and enabling seamless integration of data from legacy systems, portable devices, and modern medical technologies. The time synchronization module assigns accurate and unified timestamps to all device data, ensuring temporal alignment and resolving discrepancies caused by the inherent inaccuracies in device clocks, creating a synchronized data stream. Therefore, the synergy is that the protocol conversion ensures data from diverse devices is usable and standardized and time synchronization aligns the converted data temporally, making it possible to correlate and analyze inputs from multiple devices. For example, an arterial line pressure reading (normalized via protocol conversion) is synchronized with ECG data to enable predictive models to assess cardiovascular risks accurately in real-time.

In another embodiment, the synergy between time synchronization and predictive analytics is defined as the time synchronization module provides the temporal precision necessary for analyzing relationships between events (e.g., drug administration and physiological changes) and creates a unified timeline for cross-device data, critical for high-frequency or sub-second decision-making. The predictive analytics module uses synchronized, high-quality data to invoke AI models and expert systems to detect adverse medical events or generate actionable insights. It relies on synchronized metadata to ensure input consistency and avoid prediction errors. Therefore, the synergy is that the predictive analytics module cannot function effectively without accurately synchronized data. The unified timeline ensures that one or more AI models 906 analyze cause-and-effect relationships correctly, such as determining whether a drug administration preceded or succeeded a physiological change. For example, a sepsis prediction model uses synchronized heart rate, temperature, and oxygen saturation data to identify early warning signs with high reliability.

In another embodiment, the synergy between protocol conversion and predictive analytics is defined as the protocol conversion module standardizes diverse device data into formats compatible with existing AI models and expert systems and expands access to advanced analytics by converting third-party device outputs into actionable inputs for virtual medical device analyses or pre-trained AI systems. The predictive analytics module processes the standardized data to generate insights such as stroke volume, cardiac output, or arrhythmia detection. It incorporates outputs into patient records as new inputs for subsequent analysis. Therefore, the synergy is that the protocol conversion ensures data compatibility with AI models, while predictive analytics transforms this standardized data into clinical insights. For example, an arterial line pressure reading (converted into the required format) is processed by a virtual medical device analysis system to calculate cardiac output and stroke volume remotely.

In yet another embodiment, the synergy between contextual feedback and the core components is defined as the contextual feedback device aggregates, logs, and validates data from multiple devices, providing a complete dataset for protocol conversion and predictive analytics and functions as a local backup during network outages, ensuring data integrity and availability. The interactions with Core Components may be as follows:
With Protocol Conversion: Ensures all data, including portable or intermittent device inputs, is available in standardized formats.
With Time Synchronization: Provides the local timestamps necessary for devices lacking integrated timekeeping, ensuring that synchronized data remains consistent.
With Predictive Analytics: Augments AI models by providing additional metadata generated from the combined dataset.
   Therefore, the synergy is that the contextual feedback device enhances the reliability and depth of data available to other components, creating a more robust and integrated system. For example, portable ECG data and bedside arterial line readings are logged and validated by the contextual feedback device, synchronized with global timestamps, converted into standardized formats, and analysed by an arrhythmia detection AI system.

The interaction between components creates a system that is more than the sum of its parts. The key synergistic outcomes include a unified data pipeline as protocol conversion and time synchronization ensure that data from disparate devices is standardized and temporally aligned, forming a coherent and usable dataset for further analysis. The key synergistic outcomes include enhanced predictive power as predictive analytics leverages high-quality, synchronized, and standardized data to generate reliable insights, enhancing patient care and safety. Another key synergistic outcome includes increased Reliability and Redundancy as the contextual feedback device ensures data availability and error detection, serving as a critical support system for the other components. Another key synergistic outcome includes scalability and interoperability as the modular design allows new devices, AI models, and expert systems to be added seamlessly, ensuring the system remains adaptable to future innovations. Another key synergistic outcome includes non-obvious innovation as the interdependence of these components creates functionalities (e.g., cross-device predictive analytics, remote advanced medical analyses) that are not achievable individually, reinforcing the system's uniqueness and non-obviousness.

It may be noted that time synchronization enhances predictive analytics as time synchronization ensures a unified temporal context. Specifically, the time synchronization ensures that all data from different medical devices is assigned precise and consistent timestamps, creating a unified temporal context for analysis. The predictive analytics models rely on this unified timeline to accurately determine cause-and-effect relationships between clinical events. For example, A sepsis prediction model uses synchronized data streams from heart rate monitors, blood pressure readings, and body temperature sensors to identify early warning signs. Without synchronized timestamps, these correlations might be misaligned, leading to inaccurate predictions.

Furthermore, the time synchronization ensures high-resolution event correlation. In scenarios requiring sub-second accuracy, time synchronization ensures that rapid changes in patient vitals (e.g., oxygen saturation drops after drug administration) are accurately correlated. For example, an arrhythmia detection AI system can match irregular ECG patterns with blood pressure changes in real-time, enabling immediate clinical intervention. Also, the time synchronization ensures error reduction as synchronized data eliminates discrepancies caused by device clock inaccuracies, reducing errors in predictive analytics. For example, a ventilator may report timestamped data for oxygen delivery, while an arterial blood gas analyzer reports pH levels. Without time synchronization, these data points might appear unrelated, leading to misinterpretation of the patient's respiratory status.

It may be noted that the protocol conversion facilitates two-way communication by interfacing with legacy and modern devices. Specifically, the protocol conversion bridges the gap between older devices with proprietary communication standards and newer, interoperable systems. For example, a legacy patient monitor using RS232C communicates with the system via protocol conversion, enabling integration into modern workflows.

It may be further noted that the protocol conversion facilitates error detection. Specifically, the protocol conversion facilitates error detection by cross-device validation. By normalizing data across devices, protocol conversion allows the system to cross-validate readings, identifying discrepancies or errors. For example, a pulse oximeter reports normal oxygen saturation, but a ventilator indicates reduced oxygen delivery. Protocol conversion ensures both data streams can be compared, detecting the inconsistency. In an embodiment, the protocol conversion Facilitates error detection by error logging and reporting as protocol conversion standardizes error messages from devices, allowing them to be logged and reported in a consistent format for troubleshooting and audits. For example, if a patient monitor fails to record data due to a sensor error, the system logs the event with a clear, standardized error code for rapid resolution. In another embodiment, the protocol conversion facilitates error detection by integration with predictive analytics as the protocol conversion ensures that error data can be used as inputs for predictive analytics models, enhancing their ability to detect and mitigate risks. For example, if multiple devices report frequent communication errors, a predictive model might flag potential system-wide network issues, preventing larger failures.

The integration of time synchronization and protocol conversion creates a robust framework for advanced medical device interoperability as the time synchronization enhances predictive analytics by ensuring data temporal alignment, enabling accurate event correlation, and reducing errors and the protocol conversion facilitates two-way communication and error detection by normalizing data across devices, enabling cross-validation, and ensuring compatibility with modern systems. This synergy not only improves the reliability and accuracy of medical device operations but also enhances patient safety and clinical decision-making

It may be noted that real-time localized operations are not only pivotal for current medical systems but also serve as a key enabler for autonomous AI-driven healthcare in the future. As AI increasingly takes on decision-making roles in clinical environments, the ability to operate autonomously and locally becomes essential. This innovation represents the first-ever solution to recognize and implement this capability in a complex clinical environment, setting a precedent for what will become standard practice in the future.

This system is the first to integrate real-time localized operations within a complex clinical environment, enabling autonomous functionality and seamless interaction across diverse medical devices. By recognizing the necessity of local autonomy in critical environments, the system positions itself as intrinsically innovative, solving challenges that will become obvious to others only as the field evolves. The localized operations serve as the foundation for autonomous AI modes, where AI systems must operate independently of central servers, analyze data and make decisions in real-time, and execute and validate commands without human intervention. This capability is critical for autonomous systems managing urgent or resource-constrained scenarios, such as remote healthcare or emergency settings. Furthermore, the system's localized architecture supports AI-driven adaptability, enabling it to learn from and respond to changing conditions at the point of care. For example, in an autonomous AI mode, the system could adjust ventilator settings based on real-time analysis of vital signs without requiring external input.

In an embodiment, the real-time localized operations are designed to handle the intrinsic complexity of clinical environments, where multiple devices, data streams, and scenarios converge. This innovation bridges the gap between real-time data processing and actionable insights, something previously unaddressed in a fully autonomous context. The system anticipates the evolution of healthcare into AI-driven ecosystems, where local autonomy is not optional but essential. By integrating these capabilities now, the invention ensures it will remain relevant and foundational as autonomous AI becomes the norm. In future, local real-time operations in clinical settings-will become obvious and expected as the industry adopts AI-driven models. This system's foresight establishes it as a trailblazer, fundamentally altering how healthcare systems approach localized operations and autonomy.

Furthermore, there may be several advantages in an AI-Driven Ecosystem such as uninterrupted autonomy, scalable AI decision-making, and enhanced AI Learning. The uninterrupted autonomy enables AI systems to function autonomously even in the absence of network connectivity or external oversight. For example, during a network outage, the system continues to monitor, analyze, and adjust patient care autonomously, ensuring continuity. The scalable AI decision-making provides the infrastructure for AI to make localized, context-aware decisions, scaling across devices and environments without the need for centralized oversight. For example, AI-driven triage in disaster zones, were real-time localized operations guide device usage and patient management. The enhanced AI learning as Local data aggregation and metadata generation create a rich dataset that can be used to improve AI algorithms and decision models over time. For example, AI models learning from real-time correlations between device outputs and patient outcomes, enhancing predictive capabilities.

Therefore, this disclosure lays the groundwork for the autonomous AI-driven future of healthcare by embedding real-time localized operations into a complex clinical environment. Its ability to combine local autonomy, real-time decision-making, and robust integration across medical devices ensures that it will remain a cornerstone of future healthcare systems. By being the first to recognize and implement this in a comprehensive way, the system positions itself as pioneering and intrinsically innovative, redefining what will become the standard for AI-driven healthcare.

In an embodiment, the core objectives of the protocol conversion module may be to facilitate interoperability, ensure data reliability and accuracy, enable two-way control, enhance patient safety and security, provide Localized redundancy and context, and integrate with predictive analytics. The protocol conversion module may facilitate interoperability by Converting and normalizing disparate data protocols from diverse medical devices (legacy RS232, USB, infrared, proprietary serial) into widely accepted standards (HL7, FHIR, JSON, XML, or CDT). The protocol conversion module may ensure data reliability and accuracy by synchronizing timestamps across devices, providing a consistent, high-fidelity timeline for all measurements and events. The protocol conversion module may Enable Two-Way Control as it supports secure bidirectional communication, so commands can be issued remotely to medical devices, and the module can validate and confirm their execution. The protocol conversion module may enhance patient safety and security by performing real-time validation, checking device feedback, detecting anomalies, and protecting against unauthorized or malicious data transfers. The protocol conversion module may provide localized redundancy and context by collaborating with a contextual feedback device to capture, store, and cross-check data at the point of care-even in the absence of continuous network connectivity. The protocol conversion module may integrate with predictive analytics by supplying high-quality, well-structured data to pre-trained AI models (arrhythmia detection, sepsis prediction, drug effect analysis, etc.) and include results back into the patient record.

It may be noted that the protocol conversion module consists of both hardware and software subsystems. The hardware subsystems include a Central Processing Unit (CPU) that may be Responsible for parsing, formatting, buffering, and executing tasks. The Memory includes enough RAM/flash to handle real-time data streaming, temporary caching, and local logic. The hardware subsystems further include communication interfaces such as Multiple RJ45 Ports—One port (primary) handles power (PoE, USB power, or device-supplied power) and data to/from the MDG network. Another RJ45 port (secondary) may connect directly to the medical device using, for example, an RJ45-to-serial adapter or a proprietary connector. A tertiary port is sometimes used for legacy device pass-through or additional device chaining. The communication interfaces further include Internal Communication mapped by 3 RJ45 external connectors (depending on JIT configuration)—USB (host or client), LAN (ethernets), Serial COMs (RS232), and IR transceivers. The communication interfaces further include Internal Communication modules (RF) with internal antennas such as Bluetooth, 2.4 G Wi-Fi, 5 G Wi-Fi, Access point, and Infrastructure mode of operation on both. The communication interfaces further include onboard Security & Firewall chip that could provide cryptographic functionality, ensuring secure data exchange. The hardware subsystems further include power regulation as the protocol conversion module can be powered by PoE, USB, or the medical device's own 4.5-32V output (e.g., a 12V nurse call system). The hardware subsystems further include external support backup battery.

The software subsystems include a protocol conversion layer that includes Drivers & Parsers for each supported device protocol, Formatters to translate to standardized data (HL7, FHIR, JSON, XML, CDT), Mapper that aligns device-specific data fields (e.g., “BP-systolic” or “VentPress”) with standardized resource fields, and Full disclosure Real Time OSSI proprietary protocol for full remote multi device view and analysis.

The software subsystems include a time synchronization module for NTP/Local time source integration to maintain a high-precision clock, timestamp Injector that attaches accurate timestamps to incoming data streams, and latency compensation (optional) in high-fidelity scenarios, accounts for network delay or processing overhead. The software subsystems include a two-way command controller that includes a command interpreter that receives and validates commands from the MDG (e.g., “set ventilator pressure=16 cmH2O”), a device feedback listener that listens for the device's acknowledgment or result code, and error/exception handler that raises warnings or alerts if commands fail or produce inconsistent feedback. The software subsystems include a security & observer layer that monitors incoming/outgoing data to block unauthorized requests, especially in observer mode, and every command, data transfer, and error event is logged locally (and eventually transmitted to the MDG system 904). The software subsystems include an integration with the contextual feedback device that periodically checks with the contextual feedback device for cross-validation and merges any additional metadata (e.g., combined device insights) before sending it onward to the MDG system 904.

The operational flow of the protocol conversion module may be as follows:

### 1 Setup and Registration

1. Physical Installation
   The system 102 is attached to a medical device (e.g., infusion pump, ventilator, patient monitor).
   Specific connectors (e.g., RJ45-to-serial, USB, or custom harness) are used, determined by JIT production.
2. Boot, Auto discovery and BT wireless installation.
   Upon powering up, the system 102 detects the medical device's communication interface and loads the corresponding protocol driver.
   The system 102 registers itself with the MDG system 904, providing:
      Module ID and Serial.
      Medical Device Type (e.g., ventilator).
      Location/Bed ID if available.
3. Contextual Feedback Device Coordination (Optional)
   If the contextual feedback device is present in the same room, the system 102 and feedback device exchange registration information.
   They establish a local communications channel to share real-time data logs and to cross-check device readings.

### 2 Data Collection and Protocol Conversion

1. Device Data Capture
   The medical device sends raw data (e.g., heart rate=80 bpm, blood pressure=120/80, ventilator FiO2=0.4) via its native protocol to the system 102.
   The system's 102 Protocol Conversion Layer parses and interprets these messages.
2. Time Synchronization
   As each data message arrives, the system 102 assigns an accurate timestamp (e.g., 2025-05-01T10:15:32.200Z).
   This ensures all data lines up precisely with data streams from other devices.
3. Normalization and Packaging
   The system 102 translates the parsed data into the chosen standard (e.g., HL7 message, FHIR resource, JSON object).
   The Security & Observer Layer may attach additional metadata (e.g., encryption signatures, device ID, error codes).
4. Transmission to MDG system
   The standardized data is sent (in real time or in batches, depending on configuration) to the MDG system 904.
   Simultaneously, the contextual feedback device may record the same data locally for redundancy.

### 3 Two-Way Communication and Command Execution

1. Command Origination
   A doctor or authorized AI module in the MDG system 904 determines a need to adjust a medical device parameter (e.g., reduce ventilator pressure, change alarm thresholds).
The command is formulated in a standard format (e.g., HL7 order message, FHIR operation).
2. Command Validation
   The system's 102 two-way command controller receives the command from the MDG system 904.
   It verifies:
      Command authenticity and authorization (e.g., cryptographic signature).
      Feasibility based on known device constraints (e.g., alarm setting cannot exceed a max limit).
      Contextual checks if the contextual feedback device indicates any conflicting data or error states.
3. Device Execution
   The system 102 translates the standardized command into the native protocol of a medical device.
   The medical device either executes the action (e.g., sets alarm threshold to 100 bpm) or returns an error/acknowledgment.
4. Feedback and Confirmation
   Upon execution, the medical device sends back a status code or updated readings.
   The system 102 verifies that the device's state matches the intended command outcome (e.g., new alarm threshold recognized).
   If a mismatch arises or the device fails to acknowledge, the system 102 logs an error and notifies MDG system 904 and on-site staff.

### 4 Predictive Analytics Integration

1. Data Routing
   Once the system 102 sends standardized data to the MDG system 904, the MDG system 904 invoke relevant AI models from the one or more AI models 906 or expert systems (e.g., sepsis scoring, arrhythmia detection).
   For example, if the data includes a 3-lead ECG trace in HL7 format, the MDG system 904 may pass it to an arrhythmia detection AI model.
2. Result Incorporation
   The AI model returns a result (e.g., “high suspicion of atrial fibrillation”).
   The MDG system 904 logs the result in the patient's record.
   If immediate action is needed (e.g., changing device settings, alerting the care team), a new command is generated and sent back through the module.
3. Contextual Feedback Device Support
   The contextual feedback device can supply additional local metadata, such as patient posture or environment changes (e.g., an oxygen cylinder changed). This data can further refine AI predictions.

### 5 Localized Real-Time Operations

1. Autonomous Operation in Network Disruptions
   If network connectivity to the MDG system 904 is lost, the system 102 and contextual feedback device continue to operate autonomously, collecting and time-stamping data.
   Commands can still be executed locally by staff with physical access, and logs are stored until connectivity is restored.
2. Incongruence and Error Detection
   The contextual feedback device cross-checks multiple streams (e.g., pulse oximeter says 95% SpO2, ventilator FiO2 is 0.50 possible mismatch?).
   If an inconsistency is found, it notifies the module and generates local alerts.
3. Backup and Synchronization
   Once the network is back, all locally collected data is synchronized to the MDG, closing any gaps and maintaining a continuous record.

Therefore, the positive outcomes and benefits of the disclosure may be enhanced patient safety as accurate timestamping prevents critical misalignment of events (e.g., you know exactly when a drug was given vs. when vitals changed), and real-time error alerts as if a device fails to execute a life-saving command (e.g., adjusting ventilator pressure), staff and the MDG system 904 are immediately alerted. The disclosure provides improved interoperability as legacy devices that previously could not share data or receive remote commands are now integrated into the same modern workflow and Simplified data exchange promotes unified patient records with consistent, standard formats. The disclosure provides two-way control and reduces manual workload as the clinicians can adjust device settings from anywhere (with proper authorization) and trust that the system 102 verifies execution and less time is spent physically checking or reconfiguring devices room by room. Another positive outcome is AI-driven insights as high-quality, synchronized data significantly improves AI model accuracy (e.g., arrhythmia detection, sepsis prediction) and faster decision-making is possible since analytics can be automated and immediate. Another positive outcome is reduced errors & cybersecurity threats as the security/observer features log all traffic and can block suspicious communications and Malicious attempts to issue harmful commands to a device can be filtered at the module. Another positive outcome is autonomous future-readiness as in an AI-driven healthcare environment, local real-time operations and secure two-way communication are essential for autonomous or semi-autonomous patient care. Also, the module's design is forward-compatible with advanced AI features and device evolutions

A concrete example scenario is presented below:
1. ICU Ventilator and Infusion Pump
   Both are connected to protocol conversion modules.
   Ventilator streams airway pressure and FiO2 data; the infusion pump streams infusion rates and fluid volumes.
2. Contextual Feedback Device
   Aggregates from an ECG monitor, a portable blood pressure cuff, and a bedside nurse call system.
   Notices patient's blood pressure is low while the ventilator's FiO2 is unexpectedly high-flags possible mismatch.
3. Action and Alert
   The MDG's AI system receives data indicating potential respiratory compromise+hypotension=early sepsis sign.
   AI suggests an increase in IV fluid rate; a command is sent to the infusion pump.
   Module checks the command's validity (no existing alarm conflict, dosage within safe range), then issues it.
   Infusion pump updates its rate and sends a confirmation back.
   The module logs the result, and the contextual device cross-verifies the patient's vitals to ensure improvement.
4. Outcome
   Patient's blood pressure stabilizes; staff is relieved from repeatedly checking manual logs.
   All data and events are securely recorded, time-aligned, and available for audit or further AI analysis.

By detailing each functional step—from physical attachment and power options to data standardization, two-way commands, localized analytics, and AI integration—the protocol conversion module emerges as a comprehensive solution for modernizing clinical workflows. It fosters:
Seamless interoperability across diverse medical devices.
Trusted data integrity through precise time synchronization and local backup.
Immediate, context-aware responses to changing patient conditions or device issues.
Autonomous and AI-driven operations for the future of healthcare.

Crucially, these outcomes would not be possible without each of the module's components interacting synergistically with the MDG system 904, the contextual feedback device, and the one or more data sources 104 themselves. This holistic, well-coordinated design enables truly next-generation clinical care, improving patient outcomes and reducing the burden on healthcare providers.

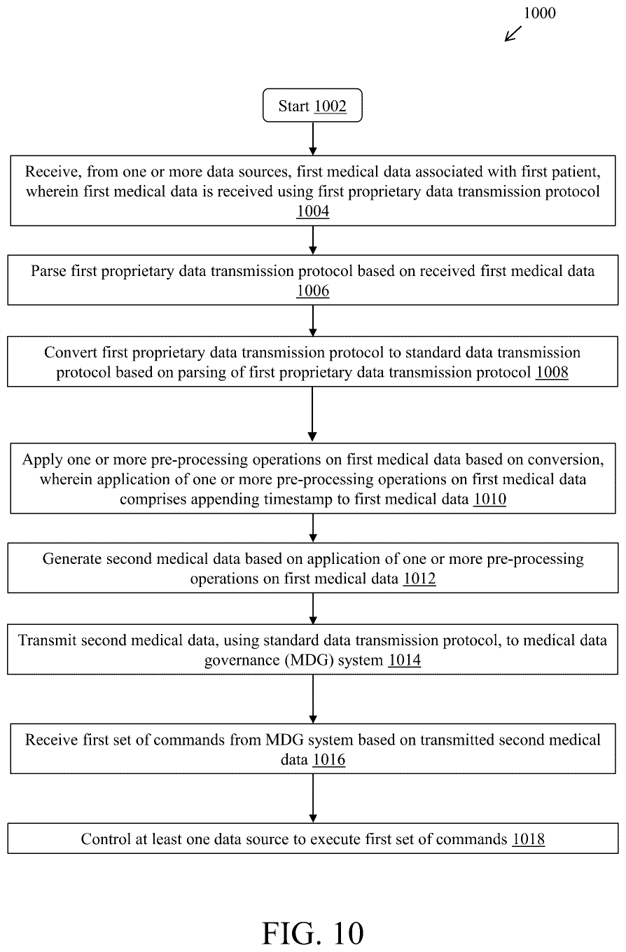

FIG. 10 is a flowchart that illustrates a method for data transfer mechanism between medical devices for medical data governance, in accordance with an embodiment of the disclosure. FIG. 10 is explained in conjunction with elements from FIGS. 1, 2, 3, 4, 5, 6, 7, 8, and 9. With reference to FIG. 10, there is shown a flowchart 1000. The operations of the exemplary method may be executed by any computing system, for example, by the system 102 of FIG. 1 or the circuitry 202 of FIG. 2. The operations of the flowchart 1000 may start at 1002 and may proceed to 1004.

At 1004, the first medical data associated with the first patient 116 may be received from one or more data sources 104. The first medical data may be received using a first proprietary data transmission protocol. In at least one embodiment, the circuitry 202 may receive, from the one or more data sources 104, the first medical data associated with the first patient 116, wherein the first medical data is received using a first proprietary data transmission protocol.

At 1006, the first proprietary data transmission protocol is parsed based on the received first medical data. In at least one embodiment, the circuitry 202 may parse the first proprietary data transmission protocol based on the received first medical data.

At 1008, the first proprietary data transmission protocol may be converted to a standard data transmission protocol based on the parsing of the first proprietary data transmission protocol. In at least one embodiment, the circuitry 202 may convert the first proprietary data transmission protocol to a standard data transmission protocol based on the parsing of the first proprietary data transmission protocol.

At 1010, one or more pre-processing operations may be applied on the first medical data based on the conversion. The application of the one or more pre-processing operations on the first medical data comprises appending a timestamp to the first medical data. In at least one embodiment, the circuitry 202 may apply one or more pre-processing operations on the first medical data based on the conversion, wherein the application of the one or more pre-processing operations on the first medical data comprises appending a timestamp to the first medical data.

At 1012, the second medical data may be generated based on the application of one or more pre-processing operations on the first medical data. In at least one embodiment, the circuitry 202 may generate second medical data based on the application of one or more pre-processing operations on the first medical data.

At 1014, the second medical data is transmitted to the medical data governance (MDG) system using the standard data transmission protocol. In at least one embodiment, the circuitry 202 may transmit the second medical data, using the standard data transmission protocol, to a medical data governance (MDG) system.

At 1016, the first set of commands may be received from the MDG system 904 based on the transmitted second medical data. In at least one embodiment, the circuitry 202 may receive a first set of commands from the MDG system 904 based on the transmitted second medical data.

At 1018, the at least one data source may be controlled to execute the first set of commands. In at least one embodiment, the circuitry 202 may control at least one data source to execute the first set of commands.

As utilized herein, the term “exemplary” means serving as a non-limiting example, instance, or illustration. As utilized herein, the terms “for example,” and “for example” set off lists of one or more non-limiting examples, instances, or illustrations. As utilized herein, circuitry is “operable” to perform a function whenever the circuitry comprises the necessary hardware and/or code (if any is necessary) to perform the function, regardless of whether the performance of the function is disabled, or not enabled, by some user-configurable setting.

The terminology used herein is for the purpose of describing particular embodiments only and is not intended to be limiting of embodiments of the disclosure. As used herein, the singular forms “a”, “an” and “the” are intended to include the plural forms as well, unless the context clearly indicates otherwise. It will be further understood that the terms “comprise”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, many embodiments are described in terms of sequences of actions to be performed by, for example, elements of a computing device. It will be recognized that various actions described herein can be performed by specific circuits (for example, application-specific integrated circuits (ASICs)), by program instructions being executed by one or more processors, or by a combination of both. Additionally, these sequences of actions described herein can be considered to be embodied entirely within any non-transitory form of computer readable storage medium having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

Another embodiment of the disclosure may provide a non-transitory machine and/or computer-readable storage and/or media, having stored thereon, a machine code and/or a computer program having at least one code section executable by a machine and/or a computer, thereby causing the machine and/or computer to perform the steps as described herein for generating a novel molecular structure using a protein structure.

The present disclosure may also be embedded in a computer program product, which comprises all the features enabling the implementation of the methods described herein, and which when loaded in a computer system is able to carry out these methods. Computer program in the present context means any expression, in any language, code or notation, either statically or dynamically defined, of a set of instructions intended to cause a system having an information processing capability to perform a particular function either directly or after either or both of the following: a) conversion to another language, code or notation; b) reproduction in a different material form.

Further, those of skill in the art will appreciate that the various illustrative logical blocks, modules, circuits, algorithms, and/or steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The methods, sequences and/or algorithms described in connection with the embodiments disclosed herein may be embodied directly in firmware, hardware, in a software module executed by a processor, or in a combination thereof. A software module may reside in RAM, flash memory, ROM memory, EPROM, EEPROM, registers, hard disk, physical and/or virtual disk, a removable disk, a CD-ROM, virtualized system or device such as a virtual server or container, or any other form of storage medium known in the art. An exemplary storage medium is communicatively coupled to the processor (including logic/code executing in the processor) such that the processor can read information from, and write information to, the storage medium. In the alternative, the storage medium may be integral to the processor.

While the present disclosure has been described with reference to certain embodiments, it will be noted understood by, for example, those skilled in the art that various changes and modifications could be made and equivalents may be substituted without departing from the scope of the present disclosure as defined, for example, in the appended claims. In addition, many modifications may be made to adapt a particular situation or material to the teachings of the present disclosure without departing from its scope. The functions, steps, and/or actions of the method claims in accordance with the embodiments of the disclosure described herein need not be performed in any particular order. Furthermore, although elements of the disclosure may be described or claimed in the singular, the plural is contemplated unless limitation to the singular is explicitly stated. Therefore, it is intended that the present disclosure is not limited to the particular embodiment disclosed, but that the present disclosure will include all embodiments falling within the scope of the appended claims.

1. A system, comprising:
a circuitry configured to:
receive, from one or more data sources, first medical data associated with a first patient, wherein the first medical data is received using a first proprietary data transmission protocol;
parse the first proprietary data transmission protocol based on the received first medical data;
convert the first proprietary data transmission protocol to a standard data transmission protocol based on the parsing of the first proprietary data transmission protocol;
apply one or more pre-processing operations on the first medical data based on the conversion, wherein the application of the one or more pre-processing operations on the first medical data comprises appending a timestamp to the first medical data;
generate second medical data based on the application of one or more pre-processing operations on the first medical data;
transmit the second medical data, using the standard data transmission protocol, to a medical data governance (MDG) system;
receive a first set of commands from the MDG system based on the transmitted second medical data; and
control at least one data source to execute the first set of commands.

2. The system according to claim 1, wherein the one or more data sources comprise at least one of: a set of medical devices, or a set of scanning devices, and wherein the set of medical devices comprises a set of diagnostic medical devices and a set of monitoring devices.

3. The system according to claim 2, wherein the first medical data correspond to at least one of: a medical professional's prescription note, a pathology report, an X-radiation (X-RAY) report, a computed tomography (CT) report, a magnetic resonance imaging (MRI) report, an ultrasound report, a cardiac catheter report, or a cardiac stress report associated with the first patient.

4. The system according to claim 1, wherein the MDG system comprises a set of medical record databases, and wherein a first medical record database of the set of medical record databases is associated with at least one of: the first patient, a first medical condition associated with the first patient, or a first medical facility associated with the first patient.

5. The system according to claim 1, wherein the MDG system comprises one or more artificial intelligence (AI) models, and wherein the circuitry is further configured to:
control the MDG system to apply the one or more AI models on the second medical data;
control the MDG system to generate the first set of commands based on the application of the one or more AI models on the second medical data; and
receive the first set of commands from the MDG system.

6. The system according to claim 5, wherein the circuitry is further configured to:
control the MDG system to determine an upcoming medical condition of the first patient based on the application of the one or more AI models on the second medical data;
receive the determined upcoming medical condition from the MDG system; and
render the upcoming medical condition.

7. The system according to claim 5, wherein the circuitry is further configured to:
receive, from the one or more data sources, third medical data associated with the first patient, wherein the second medical data is received after the execution of the first set of commands;
transmit the third medical data, using the standard data transmission protocol, to the MDG system;
control the MDG system to apply the one or more AI models on the second medical data, the third medical data, and the first set of commands;
control the MDG system to generate a second set of commands based on the application of the one or more AI models on the second medical data, the third medical data, and the first set of commands;
receive the generated second set of commands based on the application of the one or more AI models on the first medical data, the one or more commands, and the second medical data; and
control at least one data source to execute at least one command of the second set of commands.

8. The system according to claim 7, wherein the circuitry is further configured to:
compare the third medical data with pre-defined medical data; and
transmit an alert to a set of user devices based on the comparison.

9. The system according to claim 1, wherein the circuitry is further configured to:
generate medical metadata associated with the first patient based on the application of one or more pre-processing operations on the first medical data; and
render the generated medical metadata.

10. The system according to claim 1, wherein the circuitry is further configured to:
generate audit trails based on the first medical data, the second medical data, and the first set of commands; and
store the generated audit trails.

11. The system according to claim 1, wherein the circuitry is further configured to:
validate the received first set of commands based on one or more criteria; and
control at least one data source to execute the first set of commands based on the validation.

12. A method comprising:
receiving, from one or more data sources, first medical data associated with a first patient, wherein the first medical data is received using a first proprietary data transmission protocol;
parsing the first proprietary data transmission protocol based on the received first medical data;
converting the first proprietary data transmission protocol to a standard data transmission protocol based on the parsing of the first proprietary data transmission protocol;
applying one or more pre-processing operations on the first medical data based on the conversion, wherein the application of the one or more pre-processing operations on the first medical data comprises appending a timestamp to the first medical data;
generating second medical data based on the application of one or more pre-processing operations on the first medical data;
transmitting the second medical data, using the standard data transmission protocol, to a medical data governance (MDG) system;
receiving a first set of commands from the MDG system based on the transmitted second medical data; and
controlling at least one data source to execute the first set of commands.

13. The method according to claim 12, wherein the one or more data sources comprise at least one of: a set of medical devices, or a set of scanning devices, and wherein the set of medical devices comprises a set of diagnostic medical devices and a set of monitoring devices.

14. The method according to claim 13, wherein the first medical data correspond to at least one of: a medical professional's prescription note, a pathology report, an X-radiation (X-RAY) report, a computed tomography (CT) report, a magnetic resonance imaging (MRI) report, an ultrasound report, a cardiac catheter report, or a cardiac stress report associated with the first patient.

15. The method according to claim 12, wherein the MDG system comprises a set of medical record databases, and wherein a first medical record database of the set of medical record databases is associated with at least one of: the first patient, a first medical condition associated with the first patient, or a first medical facility associated with the first patient.

16. The method according to claim 10, further comprising:
receiving, from the one or more data sources, second medical data associated with the first patient, wherein the second medical data is received after the execution of the first set of commands;
controlling the MDG system to apply one or more artificial intelligence (AI) models on the second medical data;
control the MDG system to generate the first set of commands based on the application of the one or more AI models on the second medical data; and
receive the first set of commands from the MDG system.

17. The method according to claim 16, further comprising:
controlling the MDG system to determine an upcoming medical condition of the first patient based on the application of the one or more AI models on the second medical data;
receiving the determined upcoming medical condition from the MDG system; and
rendering the upcoming medical condition.

18. The method according to claim 12, further comprising:
receiving, from the one or more data sources, third medical data associated with the first patient, wherein the second medical data is received after the execution of the first set of commands;
transmitting the third medical data, using the standard data transmission protocol, to the MDG system;
controlling the MDG system to apply the one or more AI models on the second medical data, the third medical data, and the first set of commands;
controlling the MDG system to generate a second set of commands based on the application of the one or more AI models on the second medical data, the third medical data, and the first set of commands;
receiving the generated second set of commands based on the application of the one or more AI models on the first medical data, the one or more commands, and the second medical data; and
controlling at least one data source to execute at least one command of the second set of commands.

19. The method according to claim 12, further comprising:
generating audit trails based on the first medical data, the second medical data, and the first set of commands; and
storing the generated audit trails.

20. A non-transitory computer-readable medium including computer program instructions, which when executed by a system, cause the system to perform one or more operations comprising:
receiving, from one or more data sources, first medical data associated with a first patient, wherein the first medical data is received using a first proprietary data transmission protocol;
parsing the first proprietary data transmission protocol based on the received first medical data;
converting the first proprietary data transmission protocol to a standard data transmission protocol based on the parsing of the first proprietary data transmission protocol;
applying one or more pre-processing operations on the first medical data based on the conversion, wherein the application of the one or more pre-processing operations on the first medical data comprises appending a timestamp to the first medical data;
generating second medical data based on the application of one or more pre-processing operations on the first medical data;
transmitting the second medical data, using the standard data transmission protocol, to a medical data governance (MDG) system;
receiving a first set of commands from the MDG system based on the transmitted second medical data; and
controlling at least one data source to execute the first set of commands.
